(19) United States
(12) Patent Application Publication
(10) Pub. No.: US 2019/0275072 A1
(43) Pub. Date: Sep. 12, 2019

(54) PHARMACEUTICAL COMPOSITIONS COMPRISING ETEPLIRSEN

(71) Applicant: Sarepta Therapeutics, Inc., Cambridge, MA (US)

(72) Inventor: Thomas HOLT, Cambridge, MA (US)

(21) Appl. No.: 16/302,484

(22) PCT Filed: May 24, 2017
(86) PCT No.: PCT/US2017/034265

§ 371 Date: Nov. 16, 2018

Related U.S. Application Data
(60) Provisional application No. 62/340,947, filed on May 24, 2016.
(60) Provisional application No. 62/429,160, filed on Dec. 2, 2016.

(51) Int. Cl. A61K 31/7125; A61K 9/00; A61K 47/02

(52) U.S. Cl. CPC A61K 31/7125; A61K 47/02; A61K 9/0019

(57) ABSTRACT

Provided herein are pharmaceutical compositions comprising Eteplirsen. Also provided herein are methods of treating a muscle disease in a subject in need thereof, comprising administering to the subject a pharmaceutical composition of the disclosure.

### RELATED APPLICATIONS

This application claims the benefit of U.S. provisional patent application No. 62/340,947, filed May 24, 2016, and U.S. provisional patent application No. 62/429,160, filed Dec. 2, 2016, the entire contents of each of which are incorporated herein by reference.

### BACKGROUND

Antisense technology provides a means for modulating the expression of one or more specific gene products, including alternative splice products, and is uniquely useful in a number of therapeutic, diagnostic, and research applications. The principle behind antisense technology is that an antisense compound, e.g., an oligonucleotide, which hybridizes to a target nucleic acid, modulates gene expression activities such as transcription, splicing or translation through any one of a number of antisense mechanisms. The sequence specificity of antisense compounds makes them attractive as tools for target validation and gene functionalization, as well as therapeutics to selectively modulate the expression of genes involved in disease.

Duchenne muscular dystrophy (DMD) is caused by a defect in the expression of the protein dystrophin. The gene encoding the protein contains 79 exons spread out over more than 2 million nucleotides of DNA. Any exonic mutation that changes the reading frame of the exon, or introduces a stop codon, or is characterized by removal of an entire out of frame exon or exons, or duplications of one or more exons, has the potential to disrupt production of functional dystrophin, resulting in DMD.

Recent clinical trials testing the safety and efficacy of splice switching oligonucleotides (SSOs) for the treatment of DMD are based on SSO technology to induce alternative splicing of pre-mRNAs by steric blockade of the spliceosome (Cirak et al., 2011; Goemans et al., 2011; Kinali et al., 2009; van Deutekom et al., 2007). However, despite these successes, the pharmacological options available for treating DMD are limited.

Eteplirsen is a phosphorodiamidate morpholino oligomer (PMO) designed to skip exon 51 of the human dystrophin gene in patients with DMD who are amendable to exon 51 skipping to restore the read frame and produce a functional shorter form of the dystrophin protein. Sarepta Therapeutics, Inc., submitted a New Drug Application (NDA) to the United States Food and Drug Administration (FDA) seeking approval for the treatment of DMD in patients amendable to exon 51 skipping. Sarepta's NDA is currently under review by the FDA.

Although significant progress has been made in the field of antisense technology, there remains a need in the art for pharmaceutical formulations comprising oligonucleotides.

### SUMMARY

Provided herein are pharmaceutical compositions comprising Eteplirsen wherein the concentration of Eteplirsen is about 50 mg/mL of the pharmaceutical composition. Also provided herein are methods of treating a muscle disease in a subject in need thereof, comprising administering to the subject a pharmaceutical composition of the disclosure.

Accordingly, in one aspect, provided herein is a pharmaceutical composition, comprising:

a) Eteplirsen;

b) sodium chloride;

c) potassium chloride;

d) potassium phosphate monobasic;

e) sodium phosphate dibasic; and

f) water,

wherein the concentration of Eteplirsen is about 50 mg/mL of the pharmaceutical composition.

Other formulation substances that are known in general to one skilled in the art are also conceived.

In yet another aspect, provided herein is a pharmaceutical composition, comprising:

a) 40-60 mg of Eteplirsen;

b) 6.4-9.6 mg of sodium chloride;

c) 0.16-0.24 mg of potassium chloride;

d) 0.16-0.24 mg of potassium phosphate monobasic;

e) 0.91-1.37 mg of sodium phosphate dibasic; and

f) water.

In another aspect, provided herein is a pharmaceutical composition, comprising:

a) 80-120 mg of Eteplirsen;

b) 12.8-19.2 mg of sodium chloride;

c) 0.32-0.48 mg of potassium chloride;

d) 0.32-0.48 mg of potassium phosphate monobasic;

e) 1.02-1.54 mg of sodium phosphate dibasic; and

f) water.

In still another aspect, provided herein is a pharmaceutical composition, comprising:

a) 400-600 mg of Eteplirsen;

b) 64-96 mg of sodium chloride;

c) 1.6-2.4 mg of potassium chloride;

d) 1.6-2.4 mg of potassium phosphate monobasic;

e) 9.0-14.0 mg of sodium phosphate dibasic; and

f) water.

Pharmaceutical compositions of the disclosure comprise a concentration of Eteplirsen of about 50 mg/mL of the pharmaceutical composition.

In another aspect, provided herein is a method of treating a muscle disease in a subject in need thereof, comprising administering to the subject a pharmaceutical composition provided herein.

### BRIEF DESCRIPTION OF THE FIGURES

FIG. 1 shows a representative analytical high performance liquid chromatography (HPLC) chromatogram of a synthesized and deprotected Eteplirsen (AVI-4658) crude drug substance (see Example 1).

FIG. 2 shows a representative analytical HPLC chromatogram of a purified Eteplirsen drug substance solution (see Example 2).

FIG. 3 shows a representative analytical HPLC chromatogram of a desalted and lyophilized Eteplirsen drug substance (see Example 2).

### DETAILED DESCRIPTION

Provided herein are pharmaceutical compositions comprising Eteplirsen. Also provided herein are methods of treating a muscle disease in a subject in need thereof, comprising administering to the subject a pharmaceutical composition of the disclosure. The morpholino oligonucleotide described herein (Eteplirsen) displays stronger affinity for DNA and RNA without compromising sequence selectivity, relative to native or unmodified oligonucleotides. In some embodiments, the oligonucleotide of the disclosure minimizes or prevents cleavage by RNase H. In some embodiments, the antisense oligonucleotide of the disclosure does not activate RNase H.

### Definitions

Listed below are definitions of various terms used to describe this disclosure. These definitions apply to the terms as they are used throughout this specification and claims, unless otherwise limited in specific instances, either individually or as part of a larger group.

The term “support-bound” refers to a chemical moiety that is covalently linked to a support-medium.

The term “support-medium” refers to any material including, for example, any particle, bead, or surface, upon which an oligomer can be attached or synthesized upon, or can be modified for attachment or synthesis of an oligomer. Representative substrates include, but are not limited to, inorganic supports and organic supports such as glass and modified or functionalized glass, plastics (including acrylics, polystyrene and copolymers of styrene and other materials, polypropylene, polyethylene, polybutylene, polyurethanes, TEFLON, etc.), polysaccharides, nylon or nitrocellulose, ceramics, resins, silica or silica-based materials including silicon and modified silicon, carbon, metals, inorganic glasses, plastics, optical fiber bundles, and a variety of other polymers. Particularly useful solid supports and solid surfaces for some embodiments are located within a flow cell apparatus. In some embodiments of the processes described herein, the support-medium comprises polystyrene with 1% crosslinked divinylbenzene.

In some embodiments, representative support-medium comprise at least one reactive site for attachment or synthesis of an oligomer. For example, in some embodiments, a support-medium of the disclosure comprises one or more terminal amino or hydroxyl groups capable of forming a chemical bond with an incoming nucleoside or other activated group for attaching or synthesizing an oligomer.

Some representative support-medium that are amenable to the processes described herein include, but are not limited to, the following: controlled pore glass (CPG); oxalyl-controlled pore glass (see, e.g., Alul et al., Nucleic Acids Research 1991, 19, 1527); silica-containing particles, such as porous glass beads and silica gel such as that formed by the reaction of trichloro-[3-(4-chloromethyl)phenyl]propylsilane and porous glass beads (see Parr and Grohmann, Angew. Chem. Internal. Ed. 1972, 11, 314; sold under the trademark “PORASIL E” by Waters Associates, Framingham, Mass., USA); a mono ester of 1,4-dihydroxymethylbenzene and silica (see Bayer and Jung, Tetrahedron Lett. 1970, 51, 4503; sold under the trademark “BIOPAK” by Waters Associates); TENTAGEL (see, e.g., Wright et al., Tetrahedron Lett. 1993, 34, 3373); cross-linked styrene/divinylbenzene copolymer beaded matrix, or POROS, a copolymer of polystyrene/divinylbenzene (available from PerSeptive Biosystems); soluble support-medium such as polyethylene glycol PEG's (see Bonora et al., Organic Process Research & Development 2000, 4, 225-231); PEPS support, which is a polyethylene (PE) film with pendant long-chain polystyrene (PS) grafts (see Berg et al., J. Am. Chem. Soc. 1989, 111, 8024 and International Patent Application WO 1990/02749); copolymers of dimethylacrylamide cross-linked with N,N′-bisacryloylethylenediamine, including a known amount of N-tertbutoxycarbonyl-beta-alanyl-N′-acryloylhexamethylenediamine (see Atherton et al., J. Am. Chem. Soc. 1975, 97, 6584; Atherton et al., Bioorg. Chem. 1979, 8, 351; and Atherton et al., J. Chem. Soc. Perkin 11981, 538); glass particles coated with a hydrophobic cross-linked styrene polymer (see Scott et al., J. Chrom. Sci. 1971, 9, 577); fluorinated ethylene polymer onto which has been grafted polystyrene (see Kent and Merrifield, Israel J. Chem. 1978, 17, 243 and van Rietschoten in Peptides 1974, Y. Wolman, Ed., Wiley and Sons, New York, 1975, pp. 113-116); hydroxypropylacrylate-coated polypropylene membranes (Daniels et al., Tetrahedron Lett. 1989, 30, 4345); acrylic acid-grafted polyethylene-rods (Geysen et al., Proc. Natl. Acad. Sci. USA 1984, 81, 3998); a “tea bag” containing traditionally-used polymer beads (Houghten, Proc. Natl. Acad. Sci. USA 1985, 82, 5131); and combinations thereof.

The term “flow cell apparatus” refers to a chamber comprising a surface (e.g., solid surface) across which one or more fluid reagents (e.g., liquid or gas) can be flowed.

The term “treating” or “treatment” as used herein comprises a treatment relieving, reducing or alleviating at least one symptom in a subject or effecting a delay of progression of a disease. For example, treatment can be the diminishment of one or several symptoms of a disorder or complete eradication of a disorder, such as muscular dystrophy, e.g., Duchenne muscular dystrophy. Within the meaning of the present disclosure, the term “treat” also denotes to arrest, delay the onset (i.e., the period prior to clinical manifestation of a disease) or reduce the risk of developing or worsening a disease. The term “protect” is used herein to mean prevent, delay, or treat, or all, as appropriate, development, continuance or aggravation of a disease in a subject, e.g., a mammal or human. The term “prevent,” “preventing” or “prevention” as used herein comprises the prevention of at least one symptom associated with or caused by the state, disease or disorder being prevented.

The term “subject” or “patient” as used herein is intended to include animals, which are capable of suffering from or afflicted with a muscle disease or any disorder involving, directly or indirectly, a muscle disease. Examples of subjects include mammals, e.g., humans, apes, monkeys, dogs, cows, horses, pigs, sheep, goats, cats, mice, rabbits, rats, and transgenic non-human animals. In an embodiment, the subject is a human, e.g., a human suffering from, at risk of suffering from, or potentially capable of suffering from muscle diseases.

“Amendable to exon 51 skipping” as used herein with regard to a subject or patient is intended to include subjects and patients having various mutations in the dystrophin gene which are amenable to exon 51 skipping. Non-limiting examples of mutations in the following exons of the dystrophin gene are amenable to exon 51 skipping include, e.g.: 45-50, 47-50, 48-50, 49-50, 50, 52, 52-63 (Leiden Duchenne muscular dystrophy mutation database, Leiden University Medical Center, The Netherlands). Determining whether a patient has a mutation in the dystrophin gene that is amenable to exon skipping is well within the purview of one of skill in the art (see, e.g., Aartsma-Rus et al., Hum Mut 2009, 30, 293-299).

The terms “comprising” and “including” are used herein in their open-ended and non-limiting sense unless otherwise noted.

The terms “a” and “an” and “the” and similar references in the context of describing the invention (especially in the context of the following claims) are to be construed to cover both the singular and the plural, unless otherwise indicated herein or clearly contradicted by context. Where the plural form is used for compounds, salts, and the like, this is taken to mean also a single compound, salt, or the like.

The terms “about” or “approximately” are generally understood by persons knowledgeable in the relevant subject area, but in certain circumstances can mean within ±10%, or within ±5%, of a given value or range.

“USP” refers to United States Pharmacopeia, incorporated herein by reference in its entirety, and indicates that the material so identified conforms to USP specification.

“NF” refers to National Formulary, incorporated herein by reference in its entirety, and indicates that the material so identified conforms to NF specifications.

### Oligomers

Morpholino-based oligomers (including antisense oligomers) are detailed, for example, in U.S. Pat. Nos. 5,698,685, 5,217,866, 5,142,047, 5,034,506, 5,166,315, 5,185,444, 5,521,063, 5,506,337, 8,299,206, and 8,076,476, International Patent Application Publication Nos. WO/2009/064471 and WO/2012/043730, and Summerton et al., Antisense Nucleic Acid Drug Dev. 1997, 7, 187-195, each of which are hereby incorporated by reference in their entirety.

Eteplirsen (see e.g., International Patent Application Publication No. WO 2006/000057, incorporated herein by reference in its entirety) has been the subject of clinical studies to test its safety and efficacy, and clinical development is ongoing. Eteplirsen is a phosphorodiamidate morpholino (PMO) antisense oligonucleotide. The dystrophin therapeutic “Eteplirsen,” also known as “AVI-4658,” is a PMO having the base sequence 5′-CTCCAACATCAAGGAAGATGGCATTTCTAG-3′ (SEQ ID NO:1). Eteplirsen is registered under CAS Registry Number 1173755-55-9. Chemical names include: RNA, [P-deoxy-P-(dimethylamino)](2′,3′-dideoxy-2′,3′-imino-2′,3′-seco)(2′a→5′)(C-m5U-C-C-A-A-C-A-m5U-C-A-A-G-G-A-A-G-A-m5U-G-G-C-A-m5U-m5U-m5U-C-m5U-A-G) (SEQ ID NO:2), 5′-[P-[4-[[2-[2-(2-hydroxyethoxy)ethoxy] ethoxy]carbonyl]-1-piperazinyl]-N,N-dimethylphosphonamidate] and P,2′,3′-trideoxy-P-(dimethylamino)-5′-O-{P-[4-(10-hydroxy-2,5,8-trioxadecanoyl)piperazin-1-yl]-N,N-dimethylphosphonamidoyl}-2′,3′-imino-2′,3′-secocytidylyl-(2′a→5′)-P,3′-dideoxy-P-(dimethylamino)-2′,3′-imino-2′,3′-secothymidylyl-(2′a→5′)-P,2′,3′-trideoxy-P-(dimethylamino)-2′,3′-imino-2′,3′-secocytidylyl-(2′a→5′)-P,2′,3′-trideoxy-P-(dimethylamino)-2′,3′-imino-2′,3′-secocytidylyl-(2′a→5′)-P,2′,3′-trideoxy-P-(dimethylamino)-2′,3′-imino-2′,3′-secoadenylyl-(2′a→5′)-P,2′,3′-trideoxy-P-(dimethylamino)-2′,3′-imino-2′,3′-secoadenylyl-(2′a→5′)-P,2′,3′-trideoxy-P-(dimethylamino)-2′,3′-imino-2′,3′-secocytidylyl-(2′a→5′)-P,2′,3′-trideoxy-P-(dimethylamino)-2′,3′-imino-2′,3′-secoadenylyl-(2′a→5′)-P,3′-dideoxy-P-(dimethylamino)-2′,3′-imino-2′,3′-secothymidylyl-(2′a→5′)-P,2′,3′-trideoxy-P-(dimethylamino)-2′,3′-imino-2′,3′-secocytidylyl-(2′a→5′)-P,2′,3′-trideoxy-P-(dimethylamino)-2′,3′-imino-2′,3′-secoadenylyl-(2′a→5′)-P,2′,3′-trideoxy-P-(dimethylamino)-2′,3′-imino-2′,3′-secoadenylyl-(2′a→5′)-P,2′,3′-trideoxy-P-(dimethylamino)-2′,3′-imino-2′,3′-secoguanylyl-(2′a→5′)-P,2′,3′-trideoxy-P-(dimethylamino)-2′,3′-imino-2′,3′-secoguanylyl-(2′a→5′)-P,2′,3′-trideoxy-P-(dimethylamino)-2′,3′-imino-2′,3′-secoadenylyl-(2′a 5′)-P,2′,3′-trideoxy-P-(dimethylamino)-2′,3′-imino-2′,3′-secoadenylyl-(2′a→5′)-P,2′,3′-trideoxy-P-(dimethylamino)-2′,3′-imino-2′,3′-secoguanylyl-(2′a→5′)-P,2′,3′-trideoxy-P-(dimethylamino)-2′,3′-imino-2′,3′-secoadenylyl-(2′a→5′)-P,3′-dideoxy-P-(dimethylamino)-2′,3′-imino-2′,3′-secothymidylyl-(2′a→5′)-P,2′,3′-trideoxy-P-(dimethylamino)-2′,3′-imino-2′,3′-secoguanylyl-(2′a→5′)-P,2′,3′-trideoxy-P-(dimethylamino)-2′,3′-imino-2′,3′-secoguanylyl-(2′a→5′)-P,2′,3′-trideoxy-P-(dimethylamino)-2′,3′-imino-2′,3′-secocytidylyl-(2′a→5′)-P,2′,3′-trideoxy-P-(dimethylamino)-2′,3′-imino-2′,3′-secoadenylyl-(2′a 5′)-P,3′-dideoxy-P-(dimethylamino)-2′,3′-imino-2′,3′-secothymidylyl-(2′a→5′)-P,3′-dideoxy-P-(dimethylamino)-2′,3′-imino-2′,3′-secothymidylyl-(2′a→5′)-P,3′-dideoxy-P-(dimethylamino)-2′,3′-imino-2′,3′-secothymidylyl-(2′a→5′)-P,2′,3′-trideoxy-P-(dimethylamino)-2′,3′-imino-2′,3′-secocytidylyl-(2′a→5′)-P,3′-dideoxy-P-(dimethylamino)-2′,3′-imino-2′,3′-secothymidylyl-(2′a→5′)-P,2′,3′-trideoxy-P-(dimethylamino)-2′,3′-imino-2′,3′-secoadenylyl-(2′a→5′)-2′,3′-dideoxy-2′,3′-imino-2′,3′-secoguanosine.

Eteplirsen has the following structure:

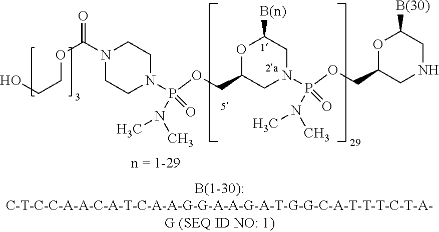

Eteplirsen can also be depicted as shown below:

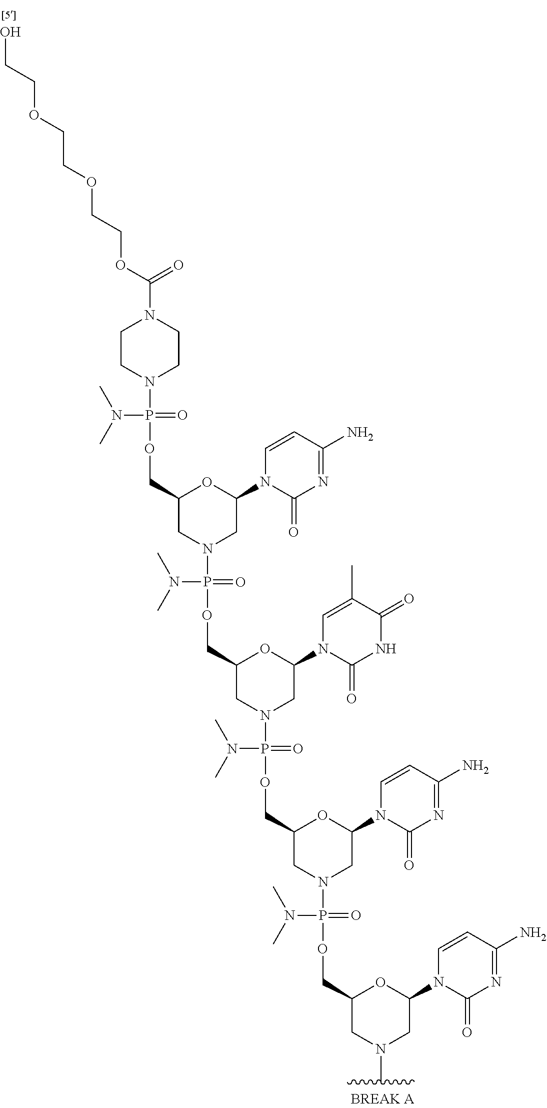 [SMILES: *N1C[C@@H](COP(=O)(N(C)C)N2C[C@@H](COP(=O)(N(C)C)N3C[C@@H](COP(=O)(N(C)C)N4C[C@@H](COP(=O)(N(C)C)N5CCN(C(=O)OCCOCCOCCO)CC5)O[C@@H](n5ccc(N)nc5=O)C4)O[C@@H](n4cc(C)c(=O)[nH]c4=O)C3)O[C@@H](n3ccc(N)nc3=O)C2)O[C@@H](n2ccc(N)nc2=O)C1]

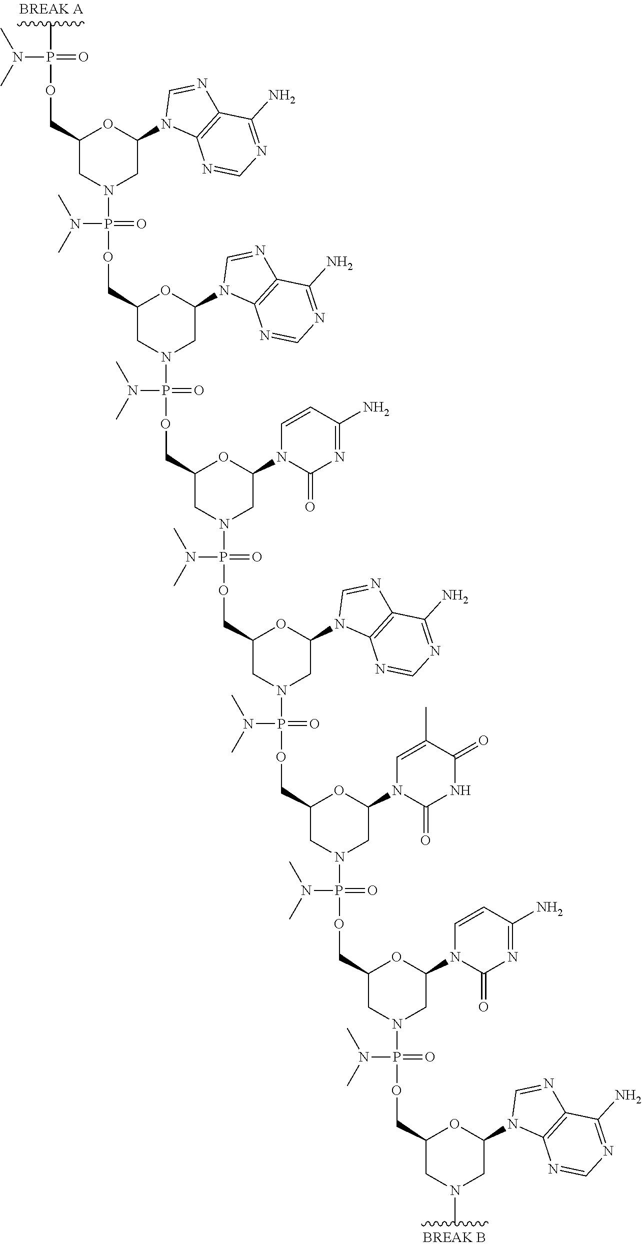 [SMILES: *N1C[C@@H](COP(=O)(N(C)C)N2C[C@@H](COP(=O)(N(C)C)N3C[C@@H](COP(=O)(N(C)C)N4C[C@@H](COP(=O)(N(C)C)N5C[C@@H](COP(=O)(N(C)C)N6C[C@@H](COP(=O)(N(C)C)N7C[C@@H](COP(*)(=O)N(C)C)O[C@@H](n8cnc9c(N)ncnc98)C7)O[C@@H](n7cnc8c(N)ncnc87)C6)O[C@@H](n6ccc(N)nc6=O)C5)O[C@@H](n5cnc6c(N)ncnc65)C4)O[C@@H](n4cc(C)c(=O)[nH]c4=O)C3)O[C@@H](n3ccc(N)nc3=O)C2)O[C@@H](n2cnc3c(N)ncnc32)C1]

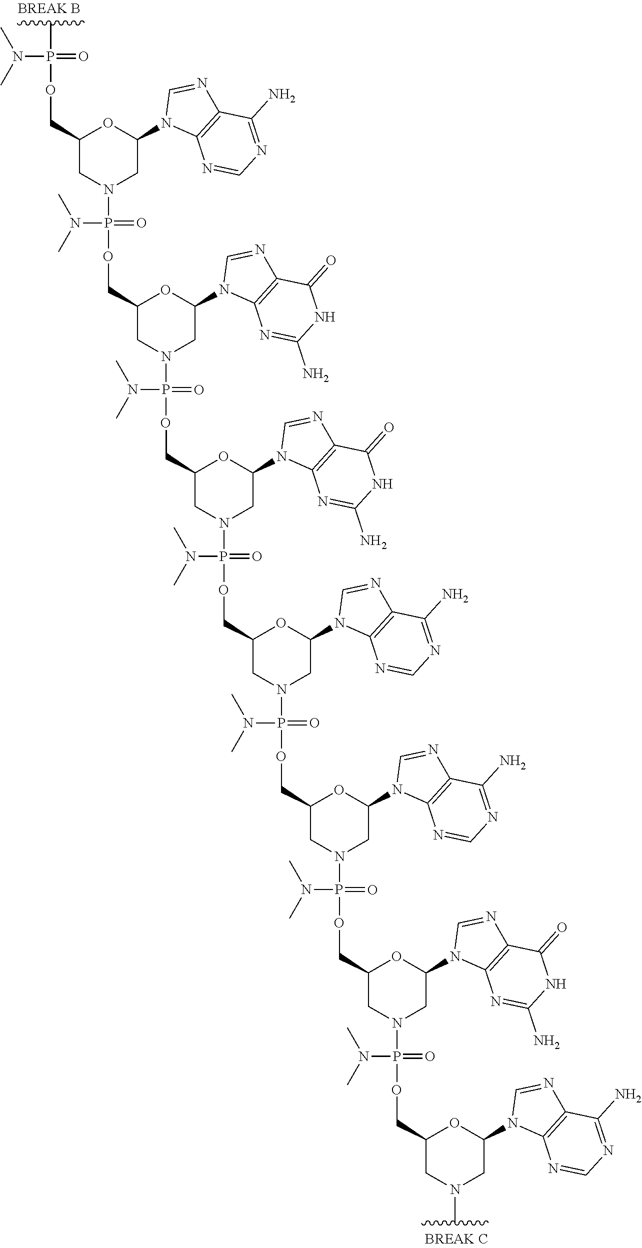 [SMILES: *N1C[C@@H](COP(=O)(N(C)C)N2C[C@@H](COP(=O)(N(C)C)N3C[C@@H](COP(=O)(N(C)C)N4C[C@@H](COP(=O)(N(C)C)N5C[C@@H](COP(=O)(N(C)C)N6C[C@@H](COP(=O)(N(C)C)N7C[C@@H](COP(*)(=O)N(C)C)O[C@@H](n8cnc9c(N)ncnc98)C7)O[C@@H](n7cnc8c(=O)[nH]c(N)nc87)C6)O[C@@H](n6cnc7c(=O)[nH]c(N)nc76)C5)O[C@@H](n5cnc6c(N)ncnc65)C4)O[C@@H](n4cnc5c(N)ncnc54)C3)O[C@@H](n3cnc4c(=O)[nH]c(N)nc43)C2)O[C@@H](n2cnc3c(N)ncnc32)C1]

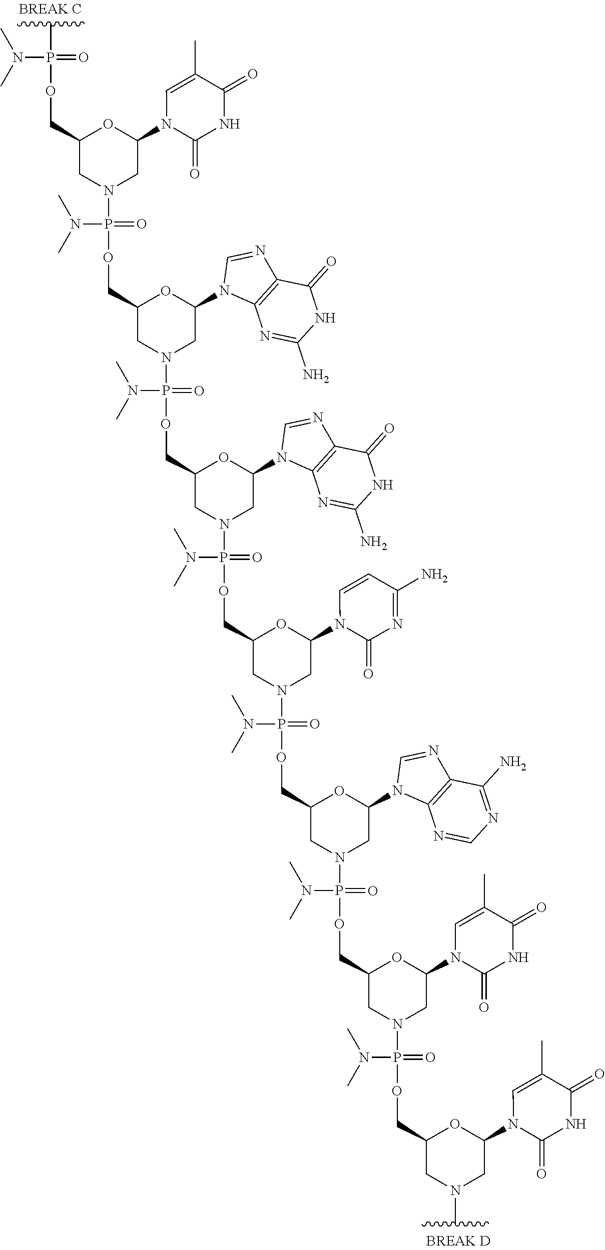 [SMILES: *N1C[C@@H](COP(=O)(N(C)C)N2C[C@@H](COP(=O)(N(C)C)N3C[C@@H](COP(=O)(N(C)C)N4C[C@@H](COP(=O)(N(C)C)N5C[C@@H](COP(=O)(N(C)C)N6C[C@@H](COP(=O)(N(C)C)N7C[C@@H](COP(*)(=O)N(C)C)O[C@@H](n8cc(C)c(=O)[nH]c8=O)C7)O[C@@H](n7cnc8c(=O)[nH]c(N)nc87)C6)O[C@@H](n6cnc7c(=O)[nH]c(N)nc76)C5)O[C@@H](n5ccc(N)nc5=O)C4)O[C@@H](n4cnc5c(N)ncnc54)C3)O[C@@H](n3cc(C)c(=O)[nH]c3=O)C2)O[C@@H](n2cc(C)c(=O)[nH]c2=O)C1]

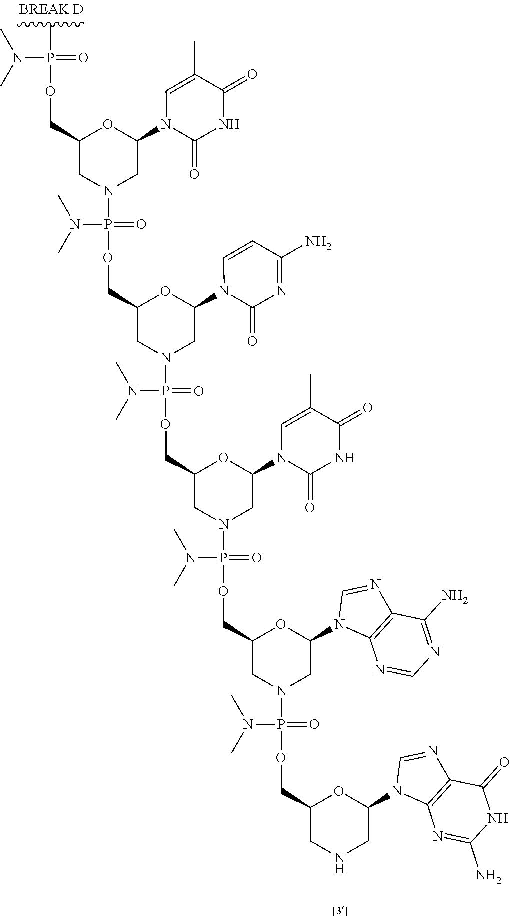 [SMILES: *P(=O)(OC[C@@H]1CN(P(=O)(OC[C@@H]2CN(P(=O)(OC[C@@H]3CN(P(=O)(OC[C@@H]4CN(P(=O)(OC[C@@H]5CNC[C@H](n6cnc7c(=O)[nH]c(N)nc76)O5)N(C)C)C[C@H](n5cnc6c(N)ncnc65)O4)N(C)C)C[C@H](n4cc(C)c(=O)[nH]c4=O)O3)N(C)C)C[C@H](n3ccc(N)nc3=O)O2)N(C)C)C[C@H](n2cc(C)c(=O)[nH]c2=O)O1)N(C)C]

For clarity, the structural formula of Eteplirsen is a continuous structural formula from 5′ to 3′, and, for the convenience of depicting the entire formula in a compact form in “BREAK A,” “BREAK B,” “BREAK C,” and “BREAK D.” As would be understood by the skilled artisan, for example, each indication of “BREAK A” shows a continuation of the illustration of the structural formula at these points. The skilled artisan understands that the same is true for each instance of “BREAK B,” “BREAK C,” and “BREAK D” in the structural formula of Eteplirsen above. None of the illustration breaks, however, are intended to indicate, nor would the skilled artisan understand them to mean, an actual discontinuation of the structural formula of Eteplirsen above.

Oligomeric compounds of the disclosure may have asymmetric centers, chiral axes, and chiral planes (as described, for example, in: E. L. Eliel and S. H. Wilen, Stereochemistry of Carbon Compounds, John Wiley & Sons, New York, 1994, pages 1119-1190, and March, J., Advanced Organic Chemistry, 3d. Ed., Chap. 4, John Wiley & Sons, New York (1985)), and may occur as racemates, racemic mixtures, and as individual diastereomers, with all possible isomers and mixtures thereof, including optical isomers. Oligomeric compounds of the disclosure herein specifically mentioned, without any indication of their stereochemistry, are intended to represent all possible isomers and mixtures thereof.

Specifically, without wishing to be bound by any particular theory, oligomeric compounds of the disclosure are prepared, as discussed herein, from activated morpholino subunits including Compound C, Compound D, Compound E, and Compound F:

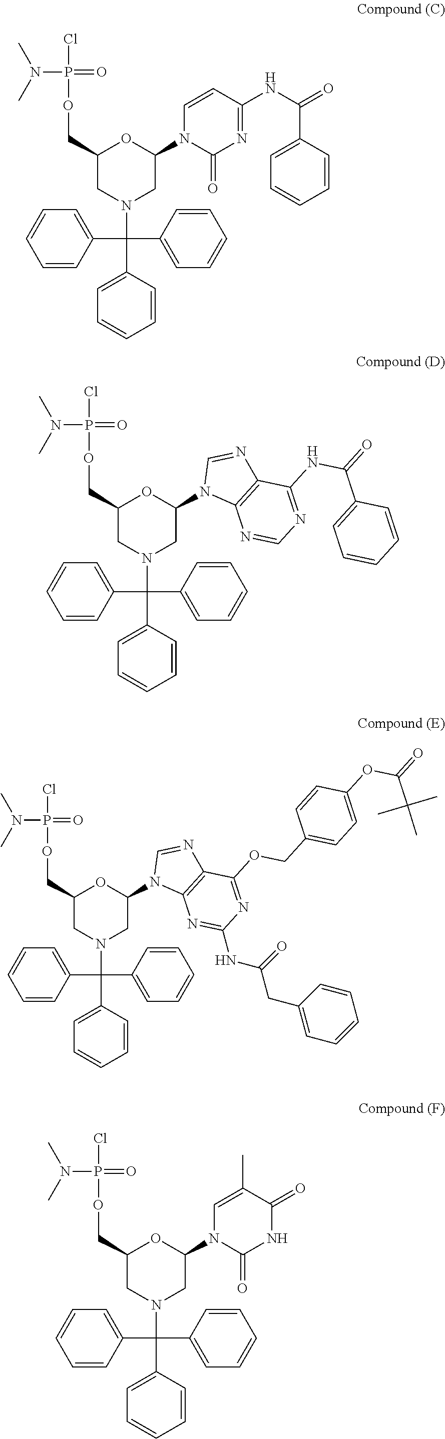

Each of Compound C, Compound D, Compound E, and Compound F may be prepared, for example, from the corresponding beta-D-ribofuranosyl as depicted below:

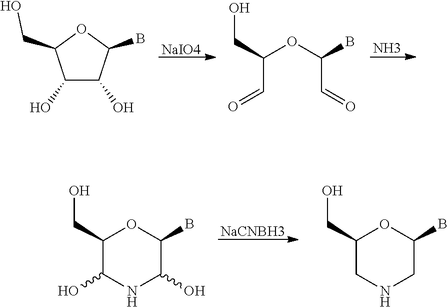

See Summerton et al., Antisense Nucleic Acid Drug Dev. 1997, 7, 187-195. Without being bound by any particular theory, the stereochemistry of the two chiral carbons is retained under the synthetic conditions. Without being bound by any particular theory, a number of possible additional stereoisomers of each morpholino subunit may otherwise be produced based on selection of, for example, an alpha-L-ribofuranosyl, alpha-D-ribofuranosyl, beta-L-ribofuranosyl, or beta-D-ribofuranosyl starting material. Without being bound by any particular theory, incorporation of 10 to 40 compounds independently selected from the group consisting of Compound C, Compound D, Compound E and Compound F, and the additional stereoisomers of each morpholino subunit for example, into an oligomeric compound may result in numerous possible stereoisomers. Without wishing to be bound by any particular theory, oligomeric compounds of the disclosure comprise one or more phosphorous-containing intersubunit linkages, which create a chiral center at each phosphorus, each of which is designated as either an “Sp” or “Rp” configuration as understood in the art. Without wishing to be bound by any particular theory, this chirality creates stereoisomers, which have identical chemical composition but different three-dimensional arrangement of their atoms. Without wishing to be bound by any particular theory, the configuration of each phosphorous intersubunit linkage occurs randomly during synthesis of, for example, oligomeric compounds of the disclosure. Without wishing to be bound by any particular theory, the synthesis process generates an exponentially large number of stereoisomers of an oligomeric compound of the disclosure because oligomeric compounds of the disclosure are comprised of numerous phosphorous-containing intersubunit linkages—with each phosphorous-containing intersubunit linkage having a random chiral configuration. Specifically, without wishing to be bound by any particular theory, each intersubunit linkage of an additional morpholino subunit doubles the number of stereoisomers of the product, so that a conventional preparation of an oligomeric compound of the disclosure is in fact a highly heterogeneous mixture of 2^{N} stereoisomers, where N represents the number of phosphorous-containing intersubunit linkages.

### Pharmaceutical Compositions

Provided herein are pharmaceutical compositions comprising Eteplirsen, or a pharmaceutically acceptable salt thereof, wherein the concentration of Eteplirsen is about 50 mg/mL of the pharmaceutical composition. In certain embodiments, the compositions are suitable for use in treating a muscle disease.

Accordingly, in one aspect, provided herein is a pharmaceutical composition, comprising:

a) Eteplirsen;

b) sodium chloride;

c) potassium chloride;

d) potassium phosphate monobasic;

e) sodium phosphate dibasic; and

f) water,

wherein the concentration of Eteplirsen is about 50 mg/mL of the pharmaceutical composition.

In yet another aspect, provided herein is a pharmaceutical composition, comprising:

a) 40-60 mg of Eteplirsen;

b) 6.4-9.6 mg of sodium chloride;

c) 0.16-0.24 mg of potassium chloride;

d) 0.16-0.24 mg of potassium phosphate monobasic;

e) 0.91-1.37 mg of sodium phosphate dibasic; and

f) water.

In one embodiment of this aspect, the pharmaceutical composition comprises about 50 mg of Eteplirsen. In another embodiment of this aspect, the total volume of the pharmaceutical composition is about 1 mL.

In another aspect, provided herein is a pharmaceutical composition, comprising:

a) about 50 mg of Eteplirsen;

b) about 8 mg of sodium chloride;

c) about 0.2 mg of potassium chloride;

d) about 0.2 mg of potassium phosphate monobasic;

e) about 1.14 mg of sodium phosphate dibasic; and

f) water.

In an embodiment of this aspect, the total volume of the pharmaceutical composition is about 1 mL.

In another aspect, provided herein is a pharmaceutical composition, comprising:

a) 50 mg of Eteplirsen;

b) 8 mg of sodium chloride;

c) 0.2 mg of potassium chloride;

d) 0.2 mg of potassium phosphate monobasic;

e) 1.14 mg of sodium phosphate dibasic; and

f) water.

In an embodiment of this aspect, the total volume of the pharmaceutical composition is 1 mL.

In another aspect, provided herein is a pharmaceutical composition, comprising:

a) 80-120 mg of Eteplirsen;

b) 12.8-19.2 mg of sodium chloride;

c) 0.32-0.48 mg of potassium chloride;

d) 0.32-0.48 mg of potassium phosphate monobasic;

e) 1.02-1.54 mg of sodium phosphate dibasic; and

f) water.

In an embodiment of this aspect, the pharmaceutical composition comprises about 100 mg of Eteplirsen. In another embodiment of this aspect, the total volume of the pharmaceutical composition is about 2 mL.

In another aspect, provided herein is a pharmaceutical composition, comprising:

a) about 100 mg of Eteplirsen;

b) about 16 mg of sodium chloride;

c) about 0.4 mg of potassium chloride;

d) about 0.4 mg of potassium phosphate monobasic;

e) about 2.28 mg of sodium phosphate dibasic; and

f) water.

In an embodiment of this aspect, the total volume of the pharmaceutical composition is about 2 mL.

In another aspect, provided herein is a pharmaceutical composition, comprising:

a) 100 mg of Eteplirsen;

b) 16 mg of sodium chloride;

c) 0.4 mg of potassium chloride;

d) 0.4 mg of potassium phosphate monobasic;

e) 2.28 mg of sodium phosphate dibasic; and

f) water.

In an embodiment of this aspect, the total volume of the pharmaceutical composition is 2 mL.

In another aspect, provided herein is a pharmaceutical composition, comprising:

a) 400-600 mg of Eteplirsen;

b) 64-96 mg of sodium chloride;

c) 1.6-2.4 mg of potassium chloride;

d) 1.6-2.4 mg of potassium phosphate monobasic;

e) 9.0-14.0 mg of sodium phosphate dibasic; and

f) water.

In an embodiment of this aspect, the pharmaceutical composition comprises about 500 mg of Eteplirsen. In another embodiment, the total volume of the pharmaceutical composition is about 10 mL.

In another aspect, provided herein is a pharmaceutical composition, comprising:

a) about 500 mg of Eteplirsen;

b) about 80 mg of sodium chloride;

c) about 2 mg of potassium chloride;

d) about 2 mg of potassium phosphate monobasic;

e) about 11.4 mg of sodium phosphate dibasic; and

f) water.

In an embodiment of this aspect, the total volume of the pharmaceutical composition is about 10 mL.

In another aspect, provided herein is a pharmaceutical composition, comprising:

a) 500 mg of Eteplirsen;

b) 80 mg of sodium chloride;

c) 2 mg of potassium chloride;

d) 2 mg of potassium phosphate monobasic;

e) 11.4 mg of sodium phosphate dibasic; and

f) water.

In an embodiment of this aspect, the total volume of the pharmaceutical composition is 10 mL.

In embodiments including, for example, some embodiments of the pharmaceutical compositions discussed above, the concentration of Eteplirsen is about 50 mg/mL of the pharmaceutical composition. In some embodiments, the concentration of Eteplirsen in the pharmaceutical composition ranges from about 45 mg/mL to about 55 mg/mL. In some embodiments, the concentration of Eteplirsen in the pharmaceutical composition ranges from 45 mg/mL to 55 mg/mL. In certain embodiments, the concentration of Eteplirsen in the pharmaceutical composition ranges from about 47.5 mg/mL to about 52.5 mg/mL. In certain embodiments, the concentration of Eteplirsen in the pharmaceutical composition ranges from 47.5 mg/mL to 52.5 mg/mL. In some embodiments, the concentration of Eteplirsen in the pharmaceutical composition is about 50 mg/mL±10%. In some embodiments, the concentration of Eteplirsen in the pharmaceutical composition is 50 mg/mL±10%. In certain embodiments, the concentration of Eteplirsen in the pharmaceutical composition is within ±10% of 50 mg/mL. In some embodiments, the concentration of Eteplirsen in the pharmaceutical composition is about 50 mg/mL±5%. In some embodiments, the concentration of Eteplirsen in the pharmaceutical composition is 50 mg/mL±5%.

In certain embodiments, the concentration of Eteplirsen in the pharmaceutical composition is within ±% of 50 mg/mL. In some embodiments, the concentration of Eteplirsen ranges from about 45.5 mg/mL to 55 mg/mL, about 46 mg/mL to about 54.5 mg/mL, about 46.5 mg/mL to about 54 mg/mL, about 47 mg/mL to about 53.5 mg/mL, about 47.5 mg/mL to about 53 mg/mL, about 45.5 mg/mL to about 52.5 mg/mL, about 45.5 mg/mL to about 52 mg/mL, about 48 mg/mL to about 51.5 mg/mL, about 48.5 mg/mL to about 51 mg/mL, about 49 mg/mL to about 50.5 mg/mL, or about 49.5 mg/mL to about 50 mg/mL of the pharmaceutical composition.

In some embodiments, the concentration of Eteplirsen in the pharmaceutical composition is about 45 mg/mL, 45.5 mg/mL, 46 mg/mL, 46.5 mg/mL, 47 mg/mL, 47.5 mg/mL, 48 mg/mL, 48.5 mg/mL, 49 mg/mL, 49.5 mg/mL, 50 mg/mL, 50.5 mg/mL, 51 mg/mL, 51.5 mg/mL, 52 mg/mL, 52.5 mg/mL, 53 mg/mL, 53.5 mg/mL, 54 mg/mL, 54.5 mg/mL, or 55 mg/mL of the pharmaceutical composition. In certain embodiments, the concentration of Eteplirsen is 45 mg/mL, 45.5 mg/mL, 46 mg/mL, 46.5 mg/mL, 47 mg/mL, 47.5 mg/mL, 48 mg/mL, 48.5 mg/mL, 49 mg/mL, 49.5 mg/mL, 50 mg/mL, 50.5 mg/mL, 51 mg/mL, 51.5 mg/mL, 52 mg/mL, 52.5 mg/mL, 53 mg/mL, 53.5 mg/mL, 54 mg/mL, 54.5 mg/mL, or 55 mg/mL of the pharmaceutical composition.

In another aspect, provided herein is a pharmaceutical composition, comprising:

a) about 5 w/v % Eteplirsen;

b) about 0.8 w/v % sodium chloride;

c) about 0.02 w/v % potassium chloride;

d) about 0.02 w/v % potassium phosphate monobasic;

e) about 0.114 w/v % sodium phosphate dibasic; and

f) water.

In an embodiment of this aspect that specifies certain w/v percentages, the total volume of the composition is 1-10 mL. In another embodiment, the total volume of the composition is about 1 mL. In another embodiment, the total volume of the composition is about 2 mL. In another embodiment, the total volume of the composition is 2 mL. In another embodiment, the total volume of the composition is about 10 mL. In another embodiment, the total volume of the composition is 10 mL.

In another embodiment of this aspect that specifies certain w/v percentages, the pharmaceutical composition comprises about 50 mg of Eteplirsen. In some embodiments, the pharmaceutical composition comprises 50 mg of Eteplirsen. In another embodiment, the pharmaceutical composition comprises about 100 mg of Eteplirsen. In another embodiment, the pharmaceutical composition comprises 100 mg of Eteplirsen. In another embodiment, the pharmaceutical composition comprises about 500 mg of Eteplirsen. In another embodiment, the pharmaceutical composition comprises 500 mg of Eteplirsen.

In another aspect, provided herein is a pharmaceutical composition, comprising:

a) 5 w/v % Eteplirsen;

b) 0.8 w/v % sodium chloride;

c) 0.02 w/v % potassium chloride;

d) 0.02 w/v % potassium phosphate monobasic;

e) 0.114 w/v % sodium phosphate dibasic; and

f) water.

In an embodiment of this aspect that specifies certain w/v percentages, the total volume of the composition is 1-10 mL. In another embodiment, the total volume of the composition is 1 mL. In another embodiment, the total volume of the composition is 2 mL. In another embodiment, the total volume of the composition is 10 mL. In another embodiment of this aspect that specifies certain w/v percentages, the pharmaceutical composition comprises 50 mg of Eteplirsen. In another embodiment, the pharmaceutical composition comprises 100 mg of Eteplirsen. In another embodiment, the pharmaceutical composition comprises 500 mg of Eteplirsen.

In another aspect, provided herein is a pharmaceutical composition, comprising:

a) about 50 mg/mL Eteplirsen;

b) about 8 mg/mL sodium chloride;

c) about 0.2 mg/mL potassium chloride;

d) about 0.2 mg/mL potassium phosphate monobasic;

e) about 1.14 mg/mL sodium phosphate dibasic; and

f) water.

In an embodiment of this aspect that specifies certain mg/mL ratios, the total volume of the composition is 1-10 mL. In another embodiment, the total volume of the composition is about 1 mL. In another embodiment, the total volume of the composition is about 2 mL. In another embodiment, the total volume of the composition is 2 mL. In another embodiment, the total volume of the composition is about 10 mL. In another embodiment, the total volume of the composition is 10 mL.

In another embodiment, the pharmaceutical composition comprises about 50 mg of Eteplirsen. In another embodiment, the pharmaceutical composition comprises 50 mg of Eteplirsen. In another embodiment, the pharmaceutical composition comprises about 100 mg of Eteplirsen. In another embodiment, the pharmaceutical composition comprises 100 mg of Eteplirsen. In another embodiment, the pharmaceutical composition comprises about 500 mg of Eteplirsen. In another embodiment, the pharmaceutical composition comprises 500 mg of Eteplirsen.

In another aspect, provided herein is a pharmaceutical composition, comprising:

a) 50 mg/mL Eteplirsen;

b) 8 mg/mL sodium chloride;

c) 0.2 mg/mL potassium chloride;

d) 0.2 mg/mL potassium phosphate monobasic;

e) 1.14 mg/mL sodium phosphate dibasic; and

f) water.

In an embodiment of this aspect that specifies certain mg/mL ratios, the total volume of the composition is 1-10 mL. In another embodiment, the total volume of the composition is 1 mL. In another embodiment, the total volume of the composition is 2 mL. In another embodiment, the total volume of the composition is 10 mL. In another embodiment, the pharmaceutical composition comprises 50 mg of Eteplirsen. In another embodiment, the pharmaceutical composition comprises 100 mg of Eteplirsen. In another embodiment, the pharmaceutical composition comprises 500 mg of Eteplirsen.

In another aspect, provided herein is a pharmaceutical composition, comprising:

a) about 50 mg of Eteplirsen;

b) about 8 mg of sodium chloride;

c) about 0.2 mg of potassium chloride;

d) about 0.2 mg of potassium phosphate monobasic;

e) about 1.14 mg of sodium phosphate dibasic; and

f) water,

wherein the total volume of the pharmaceutical composition is about 1 mL.

In another aspect, provided herein is a pharmaceutical composition, comprising:

a) 50 mg of Eteplirsen;

b) 8 mg of sodium chloride;

c) 0.2 mg of potassium chloride;

d) 0.2 mg of potassium phosphate monobasic;

e) 1.14 mg of sodium phosphate dibasic; and

f) water,

wherein the total volume of the pharmaceutical composition is 1 mL.

In another aspect, provided herein is a pharmaceutical composition, comprising:

a) about 100 mg of Eteplirsen;

b) about 16 mg of sodium chloride;

c) about 0.4 mg of potassium chloride;

d) about 0.4 mg of potassium phosphate monobasic;

e) about 2.28 mg of sodium phosphate dibasic; and

f) water,

wherein the total volume of the pharmaceutical composition is about 2 mL.

In another aspect, provided herein is a pharmaceutical composition, comprising:

a) 100 mg of Eteplirsen;

b) 16 mg of sodium chloride;

c) 0.4 mg of potassium chloride;

d) 0.4 mg of potassium phosphate monobasic;

e) 2.28 mg of sodium phosphate dibasic; and

f) water,

wherein the total volume of the pharmaceutical composition is 2 mL.

In another aspect, provided herein is a pharmaceutical composition, comprising:

a) about 500 mg of Eteplirsen;

b) about 80 mg of sodium chloride;

c) about 2 mg of potassium chloride;

d) about 2 mg of potassium phosphate monobasic;

e) about 11.4 mg of sodium phosphate dibasic; and

f) water,

wherein the total volume of the pharmaceutical composition is about 10 mL.

In another aspect, provided herein is a pharmaceutical composition, comprising:

a) 500 mg of Eteplirsen;

b) 80 mg of sodium chloride;

c) 2 mg of potassium chloride;

d) 2 mg of potassium phosphate monobasic;

e) 11.4 mg of sodium phosphate dibasic; and

f) water,

wherein the total volume of the pharmaceutical composition is 10 mL.

In another aspect, provided herein is a pharmaceutical composition, comprising:

a) 50 mg of Eteplirsen;

b) 8 mg of sodium chloride, USP;

c) 0.2 mg of potassium chloride, USP;

d) 0.2 mg of potassium phosphate monobasic, NF;

e) 1.14 mg of sodium phosphate dibasic anhydrous, USP; and

f) water for injection, USP,

wherein the total volume of the pharmaceutical composition is 1 mL, the pH of the pharmaceutical composition is about 7.5, and the osmolality of the pharmaceutical composition ranges from about 260 mOsm to about 320 mOsm.

In another aspect, provided herein is a pharmaceutical composition, comprising:

a) 100 mg of Eteplirsen;

b) 16 mg of sodium chloride, USP;

c) 0.4 mg of potassium chloride, USP;

d) 0.4 mg of potassium phosphate monobasic, NF;

e) 2.28 mg of sodium phosphate dibasic anhydrous, USP; and

f) water for injection, USP,

wherein the total volume of the pharmaceutical composition is 2 mL, the pH of the pharmaceutical composition is about 7.5, and the osmolality of the pharmaceutical composition ranges from about 260 mOsm to about 320 mOsm.

In another aspect, provided herein is a pharmaceutical composition, comprising:

a) 500 mg of Eteplirsen;

b) 80 mg of sodium chloride, USP;

c) 2 mg of potassium chloride, USP;

d) 2 mg of potassium phosphate monobasic, NF;

e) 11.4 mg of sodium phosphate dibasic anhydrous, USP; and

f) water for injection, USP,

wherein the total volume of the pharmaceutical composition is 10 mL, the pH of the pharmaceutical composition is about 7.5, and the osmolality of the pharmaceutical composition ranges from about 260 mOsm to about 320 mOsm.

In another aspect, provided herein is a pharmaceutical composition, comprising:

a) 50 mg of Eteplirsen;

b) 8 mg of sodium chloride;

c) 0.2 mg of potassium chloride;

d) 0.2 mg of potassium phosphate monobasic;

e) 1.14 mg of sodium phosphate dibasic anhydrous; and

f) water for injection,

wherein the total volume of the pharmaceutical composition is 1 mL, the pH of the pharmaceutical composition is about 7.5, and the osmolality of the pharmaceutical composition ranges from about 260 mOsm to about 320 mOsm.

In another aspect, provided herein is a pharmaceutical composition, comprising:

a) 100 mg of Eteplirsen;

b) 16 mg of sodium chloride;

c) 0.4 mg of potassium chloride;

d) 0.4 mg of potassium phosphate monobasic;

e) 2.28 mg of sodium phosphate dibasic anhydrous; and

f) water for injection,

wherein the total volume of the pharmaceutical composition is 2 mL, the pH of the pharmaceutical composition is about 7.5, and the osmolality of the pharmaceutical composition ranges from about 260 mOsm to about 320 mOsm.

In another aspect, provided herein is a pharmaceutical composition, comprising:

a) 500 mg of Eteplirsen;

b) 80 mg of sodium chloride;

c) 2 mg of potassium chloride;

d) 2 mg of potassium phosphate monobasic;

e) 11.4 mg of sodium phosphate dibasic anhydrous; and

f) water for injection,

wherein the total volume of the pharmaceutical composition is 10 mL, the pH of the pharmaceutical composition is about 7.5, and the osmolality of the pharmaceutical composition ranges from about 260 mOsm to about 320 mOsm.

In some embodiments including, for example, some embodiments discussed above, the pH of the pharmaceutical composition is about 7.5 or is 7.5. In some embodiments, the pH of the pharmaceutical composition is adjusted to about pH 7.5 with NaOH, NF, HCl, NF, or a combination thereof.

In certain embodiments including, for example, some embodiments discussed above, the osmolality of the pharmaceutical composition ranges from about 260 mOsm to about 320 mOsm. In some embodiments, the pH of the pharmaceutical composition is about 7.5 and the pharmaceutical composition ranges from about 260 mOsm to about 320 mOsm.

In a further embodiment, pharmaceutical compositions of the disclosure may additionally comprise a carbohydrate as provided in Han et al., Nat. Comms. 2016, 7, 10981, the entirety of which is incorporated herein by reference. In some embodiments, pharmaceutical compositions of the disclosure may comprise 5% of a hexose carbohydrate.

For example, pharmaceutical composition of the disclosure may comprise 5% glucose, 5% fructose, or 5% mannose. In certain embodiments, pharmaceutical compositions of the disclosure may comprise 2.5% glucose and 2.5% fructose. In some embodiments, pharmaceutical compositions of the disclosure may comprises a carbohydrate selected from: arabinose present in an amount of 5% by volume, glucose present in an amount of 5% by volume, sorbitol present in an amount of 5% by volume, galactose present in an amount of 5% by volume, fructose present in an amount of 5% by volume, xylitol present in an amount of 5% by volume, mannose present in an amount of 5% by volume, a combination of glucose and fructose each present in an amount of 2.5% by volume, and a combination of glucose present in an amount of 5.7% by volume, fructose present in an amount of 2.86% by volume, and xylitol present in an amount of 1.4% by volume.

### Methods

Provided herein are methods of treating a muscle disease in a subject in need thereof, comprising administering to the subject a pharmaceutical composition of the disclosure.

Accordingly, in one aspect, provided herein is a method of treating a muscle disease in a subject in need thereof, comprising administering to the subject a pharmaceutical composition disclosed herein. In one embodiment, the muscle disease is Duchenne muscular dystrophy.

In another aspect, provided herein is a method of preventing a muscle disease in a subject in need thereof, comprising administering to the subject a pharmaceutical composition disclosed herein. In one embodiment, the muscle disease is Duchenne muscular dystrophy.

In an additional aspect, provided herein is a method for treating Duchenne muscular dystrophy in a subject in need thereof wherein the subject has a mutation of the dystrophin gene that is amenable to exon 51 skipping, comprising administering to the subject a pharmaceutical composition of the disclosure.

The subject considered herein is typically a human. However, the subject can be any mammal for which treatment is desired. Thus, the methods described herein can be applied to both human and veterinary applications.

It will be appreciated that pharmaceutical compositions provided herein may be administered by any means known in the art. The pharmaceutical compositions provided herein are more preferably delivered by intravenous, intra-arterial, intraperitoneal, intramuscular, or subcutaneous routes of administration.

Accordingly, in one aspect, methods of the disclosure comprise administering to the subject a pharmaceutical composition, the pharmaceutical composition comprising:

a) 40-60 mg of Eteplirsen;

b) 6.4-9.6 mg of sodium chloride;

c) 0.16-0.24 mg of potassium chloride;

d) 0.16-0.24 mg of potassium phosphate monobasic;

e) 0.91-1.37 mg of sodium phosphate dibasic; and

f) water.

In one embodiment of this method, the pharmaceutical composition comprises about 50 mg of Eteplirsen. In another embodiment of this method, the total volume of the pharmaceutical composition is about 1 mL.

In another aspect, methods of the disclosure comprise administering to the subject a pharmaceutical composition, the pharmaceutical composition comprising:

a) about 50 mg of Eteplirsen;

b) about 8 mg of sodium chloride;

c) about 0.2 mg of potassium chloride;

d) about 0.2 mg of potassium phosphate monobasic;

e) about 1.14 mg of sodium phosphate dibasic; and

f) water.

In an embodiment of this method, the total volume of the pharmaceutical composition is about 1 mL.

In another aspect, methods of the disclosure comprise administering to the subject a pharmaceutical composition, the pharmaceutical composition comprising:

a) 80-120 mg of Eteplirsen;

b) 12.8-19.2 mg of sodium chloride;

c) 0.32-0.48 mg of potassium chloride;

d) 0.32-0.48 mg of potassium phosphate monobasic;

e) 1.02-1.54 mg of sodium phosphate dibasic; and

f) water.

In an embodiment of this method, the pharmaceutical composition comprises about 100 mg of Eteplirsen. In another embodiment of this method, the total volume of the pharmaceutical composition is about 2 mL. In another embodiment of this method, the total volume of the pharmaceutical composition is 2 mL.

In another aspect, methods of the disclosure comprise administering to the subject a pharmaceutical composition, the pharmaceutical composition comprising:

a) about 100 mg of Eteplirsen;

b) about 16 mg of sodium chloride;

c) about 0.4 mg of potassium chloride;

d) about 0.4 mg of potassium phosphate monobasic;

e) about 2.28 mg of sodium phosphate dibasic; and

f) water.

In an embodiment of this method, the total volume of the pharmaceutical composition is about 2 mL. In an embodiment of this method, the total volume of the pharmaceutical composition is 2 mL.

In another aspect, methods of the disclosure comprise administering to the subject a pharmaceutical composition, the pharmaceutical composition comprising:

a) 400-600 mg of Eteplirsen;

b) 64-96 mg of sodium chloride;

c) 1.6-2.4 mg of potassium chloride;

d) 1.6-2.4 mg of potassium phosphate monobasic;

e) 9.0-14.0 mg of sodium phosphate dibasic; and

f) water.

In an embodiment of this method, the pharmaceutical composition comprises about 500 mg of Eteplirsen. In another embodiment, the total volume of the pharmaceutical composition is about 10 mL. In an embodiment of this method, the pharmaceutical composition comprises 500 mg of Eteplirsen. In another embodiment, the total volume of the pharmaceutical composition is 10 mL.

In another aspect, methods of the disclosure comprise administering to the subject a pharmaceutical composition, the pharmaceutical composition comprising:

a) about 500 mg of Eteplirsen;

b) about 80 mg of sodium chloride;

c) about 2 mg of potassium chloride;

d) about 2 mg of potassium phosphate monobasic;

e) about 11.4 mg of sodium phosphate dibasic; and

f) water.

In an embodiment of this method, the total volume of the pharmaceutical composition is about 10 mL. In an embodiment of this method, the total volume of the pharmaceutical composition is 10 mL.

In embodiments including, for example, some embodiments of the pharmaceutical compositions discussed above, the concentration of Eteplirsen is about 50 mg/mL of the pharmaceutical composition. In some embodiments, the concentration of Eteplirsen in the pharmaceutical composition ranges from about 45 mg/mL to about 55 mg/mL. In some embodiments, the concentration of Eteplirsen in the pharmaceutical composition ranges from 45 mg/mL to 55 mg/mL. In certain embodiments, the concentration of Eteplirsen in the pharmaceutical composition ranges from about 47.5 mg/mL to about 52.5 mg/mL. In certain embodiments, the concentration of Eteplirsen in the pharmaceutical composition ranges from 47.5 mg/mL to 52.5 mg/mL. For example, the concentration of Eteplirsen in the pharmaceutical composition ranges from

In some embodiments, the concentration of Eteplirsen in the pharmaceutical composition is about 50 mg/mL±10%. In some embodiments, the concentration of Eteplirsen in the pharmaceutical composition is 50 mg/mL±10%. In certain embodiments, the concentration of Eteplirsen in the pharmaceutical composition is within ±10% of 50 mg/mL. In some embodiments, the concentration of Eteplirsen in the pharmaceutical composition is about 50 mg/mL±5%. In some embodiments, the concentration of Eteplirsen in the pharmaceutical composition is 50 mg/mL±5%.

In certain embodiments, the concentration of Eteplirsen in the pharmaceutical composition is within ±5% of 50 mg/mL. In some embodiments, the concentration of Eteplirsen ranges from about 45.5 mg/mL to 55 mg/mL, about 46 mg/mL to about 54.5 mg/mL, about 46.5 mg/mL to about 54 mg/mL, about 47 mg/mL to about 53.5 mg/mL, about 47.5 mg/mL to about 53 mg/mL, about 45.5 mg/mL to about 52.5 mg/mL, about 45.5 mg/mL to about 52 mg/mL, about 48 mg/mL to about 51.5 mg/mL, about 48.5 mg/mL to about 51 mg/mL, about 49 mg/mL to about 50.5 mg/mL, or about 49.5 mg/mL to about 50 mg/mL of the pharmaceutical composition.

In some embodiments, the concentration of Eteplirsen in the pharmaceutical composition is about 45.5 mg/mL, 46 mg/mL, 46.5 mg/mL, 47 mg/mL, 47.5 mg/mL, 48 mg/mL, 48.5 mg/mL, 49 mg/mL, 49.5 mg/mL, 50 mg/mL, 50.5 mg/mL, 51 mg/mL, 51.5 mg/mL, 52 mg/mL, 52.5 mg/mL, 53 mg/mL, 53.5 mg/mL, 54 mg/mL, 54.5 mg/mL, or 55 mg/mL of the pharmaceutical composition. In certain embodiments, the concentration of Eteplirsen is 45 mg/mL, 45.5 mg/mL, 46 mg/mL, 46.5 mg/mL, 47 mg/mL, 47.5 mg/mL, 48 mg/mL, 48.5 mg/mL, 49 mg/mL, 49.5 mg/mL, 50 mg/mL, 50.5 mg/mL, 51 mg/mL, 51.5 mg/mL, 52 mg/mL, 52.5 mg/mL, 53 mg/mL, 53.5 mg/mL, 54 mg/mL, 54.5 mg/mL, or 55 mg/mL of the pharmaceutical composition.

In another aspect, methods of the disclosure comprise administering to the subject a pharmaceutical composition, the pharmaceutical composition comprising:

a) about 5 w/v % Eteplirsen;

b) about 0.8 w/v % sodium chloride;

c) about 0.02 w/v % potassium chloride;

d) about 0.02 w/v % potassium phosphate monobasic;

e) about 0.114 w/v % sodium phosphate dibasic; and

f) water.

In an embodiment of this method that specifies certain w/v percentages, the total volume of the composition is 1-10 mL. In another embodiment, the total volume of the composition is about 1 mL. In another embodiment, the total volume of the composition is about 2 mL. In another embodiment, the total volume of the composition is 2 mL. In another embodiment, the total volume of the composition is about 10 mL. In another embodiment, the total volume of the composition is 10 mL.

In another embodiment of this method that specifies certain w/v percentages, the pharmaceutical composition comprises about 50 mg of Eteplirsen. In another embodiment, the pharmaceutical composition comprises 50 mg of Eteplirsen. In another embodiment, the pharmaceutical composition comprises about 100 mg of Eteplirsen. In another embodiment, the pharmaceutical composition comprises 100 mg of Eteplirsen. In another embodiment, the pharmaceutical composition comprises about 500 mg of Eteplirsen. In another embodiment, the pharmaceutical composition comprises 500 mg of Eteplirsen.

In another aspect, methods of the disclosure comprise administering to the subject a pharmaceutical composition, the pharmaceutical composition comprising:

a) 5 w/v % Eteplirsen;

b) 0.8 w/v % sodium chloride;

c) 0.02 w/v % potassium chloride;

d) 0.02 w/v % potassium phosphate monobasic;

e) 0.114 w/v % sodium phosphate dibasic; and

f) water.

In an embodiment of this aspect that specifies certain w/v percentages, the total volume of the composition is 1-10 mL. In another embodiment, the total volume of the composition is 1 mL. In another embodiment, the total volume of the composition is 2 mL.

In another embodiment, the total volume of the composition is 10 mL. In another embodiment of this aspect that specifies certain w/v percentages, the pharmaceutical composition comprises 50 mg of Eteplirsen. In another embodiment, the pharmaceutical composition comprises 100 mg of Eteplirsen. In another embodiment, the pharmaceutical composition comprises 500 mg of Eteplirsen.

In another embodiment of this method that specifies certain w/v percentages, the pharmaceutical composition comprises about 50 mg of Eteplirsen. In another embodiment, the pharmaceutical composition comprises 50 mg of Eteplirsen. In another embodiment, the pharmaceutical composition comprises about 100 mg of Eteplirsen. In another embodiment, the pharmaceutical composition comprises 100 mg of Eteplirsen. In another embodiment, the pharmaceutical composition comprises about 500 mg of Eteplirsen. In another embodiment, the pharmaceutical composition comprises 500 mg of Eteplirsen.

In another aspect, methods of the disclosure comprise administering to the subject a pharmaceutical composition, the pharmaceutical composition comprising:

a) about 50 mg/mL Eteplirsen;

b) about 8 mg/mL sodium chloride;

c) about 0.2 mg/mL potassium chloride;

d) about 0.2 mg/mL potassium phosphate monobasic;

e) about 1.14 mg/mL sodium phosphate dibasic; and

f) water.

In an embodiment of this aspect that specifies certain mg/mL ratios, the total volume of the composition is 1-10 mL. In another embodiment, the total volume of the composition is about 1 mL. In another embodiment, the total volume of the composition is about 2 mL. In another embodiment, the total volume of the composition is about 10 mL. In another embodiment, the pharmaceutical composition comprises about 50 mg of Eteplirsen. In another embodiment, the pharmaceutical composition comprises about 100 mg of Eteplirsen. In another embodiment, the pharmaceutical composition comprises about 500 mg of Eteplirsen.

In another aspect, methods of the disclosure comprise administering to the subject a pharmaceutical composition, the pharmaceutical composition comprising:

a) 50 mg/mL Eteplirsen;

b) 8 mg/mL sodium chloride;

c) 0.2 mg/mL potassium chloride;

d) 0.2 mg/mL potassium phosphate monobasic;

e) 1.14 mg/mL sodium phosphate dibasic; and

f) water.

In an embodiment of this aspect that specifies certain mg/mL ratios, the total volume of the composition is 1-10 mL. In another embodiment, the total volume of the composition is 1 mL. In another embodiment, the total volume of the composition is 2 mL. In another embodiment, the total volume of the composition is 10 mL. In another embodiment, the pharmaceutical composition comprises 50 mg of Eteplirsen. In another embodiment, the pharmaceutical composition comprises 100 mg of Eteplirsen. In another embodiment, the pharmaceutical composition comprises 500 mg of Eteplirsen.

In another aspect, methods of the disclosure comprise administering to the subject a pharmaceutical composition, the pharmaceutical composition comprising:

a) 50 mg of Eteplirsen;

b) 8 mg of sodium chloride;

c) 0.2 mg of potassium chloride;

d) 0.2 mg of potassium phosphate monobasic;

e) 1.14 mg of sodium phosphate dibasic; and

f) water,

wherein the total volume of the pharmaceutical composition is 1 mL.

In another aspect, methods of the disclosure comprise administering to the subject a pharmaceutical composition, the pharmaceutical composition comprising:

a) 100 mg of Eteplirsen;

b) 16 mg of sodium chloride;

c) 0.4 mg of potassium chloride;

d) 0.4 mg of potassium phosphate monobasic;

e) 2.28 mg of sodium phosphate dibasic; and

f) water,

wherein the total volume of the pharmaceutical composition is 2 mL.

In another aspect, methods of the disclosure comprise administering to the subject a pharmaceutical composition, the pharmaceutical composition comprising:

a) 500 mg of Eteplirsen;

b) 80 mg of sodium chloride;

c) 2 mg of potassium chloride;

d) 2 mg of potassium phosphate monobasic;

e) 11.4 mg of sodium phosphate dibasic; and

f) water,

wherein the total volume of the pharmaceutical composition is 10 mL.

In some embodiments including, for example, some embodiments discussed above, the pH of the pharmaceutical composition is about 7.5 or is 7.5. In some embodiments, the pH of the pharmaceutical composition is adjusted to about pH 7.5 with NaOH, NF, HCl, NF, or a combination thereof.

In certain embodiments including, for example, some embodiments discussed above, the osmolality of the pharmaceutical composition ranges from about 260 mOsm to about 320 mOsm. In some embodiments, the pH of the pharmaceutical composition is about 7.5 and the pharmaceutical composition ranges from about 260 mOsm to about 320 mOsm.

In a further embodiment, the pharmaceutical compositions of the disclosure may be co-administered with a carbohydrate in the methods of the disclosure, either in the same formulation or is a separate formulation, as provided in Han et al., Nat. Comms. 2016, 7, 10981, the entirety of which is incorporated herein by reference. In some embodiments, pharmaceutical compositions of the disclosure may be co-administered with 5% of a hexose carbohydrate. For example, pharmaceutical compositions of the disclosure may be co-administered with 5% glucose, 5% fructose, or 5% mannose. In certain embodiments, pharmaceutical compositions of the disclosure may be co-administered with 2.5% glucose and 2.5% fructose. In some embodiments, pharmaceutical composition of the disclosure may be co-administered with a carbohydrate selected from: arabinose present in an amount of 5% by volume, glucose present in an amount of 5% by volume, sorbitol present in an amount of 5% by volume, galactose present in an amount of 5% by volume, fructose present in an amount of 5% by volume, xylitol present in an amount of 5% by volume, mannose present in an amount of 5% by volume, a combination of glucose and fructose each present in an amount of 2.5% by volume, and a combination of glucose present in an amount of 5.7% by volume, fructose present in an amount of 2.86% by volume, and xylitol present in an amount of 1.4% by volume.

### Kits

In other embodiments, kits are provided. Kits according to the disclosure include package(s) comprising Eteplirsen, or pharmaceutical compositions of the disclosure. In some embodiments, kits comprise Eteplirsen, or a pharmaceutically acceptable salt thereof.

The phrase “package” means any vessel containing oligonucleotides or compositions presented herein. In some embodiments, the package can be a box or wrapping. Packaging materials for use in packaging pharmaceutical products are well-known to those of skill in the art. Examples of pharmaceutical packaging materials include, but are not limited to, bottles, tubes, inhalers, pumps, bags, vials, containers, syringes, bottles, and any packaging material suitable for a selected formulation and intended mode of administration and treatment.

The kit can also contain items that are not contained within the package, but are attached to the outside of the package, for example, pipettes.

Kits can further contain instructions for administering Eteplirsen or pharmaceutical compositions of the disclosure to a patient. Kits also can comprise instructions for approved uses of Eteplirsen by regulatory agencies, such as the United States Food and Drug Administration. Kits can also contain labeling or product inserts for Eteplirsen. The package(s) or any product insert(s), or both, may themselves be approved by regulatory agencies. The kits can include Eteplirsen in the solid phase or in a liquid phase (such as buffers provided) in a package. The kits can also include buffers for preparing solutions for conducting the methods, and pipettes for transferring liquids from one container to another.

### EXAMPLES

Examples have been set forth below for the purpose of illustration and to describe certain specific embodiments of the disclosure. However, the scope of the claims is not to be in any way limited by the examples set forth herein. Various changes and modifications to the disclosed embodiments will be apparent to those skilled in the art and such changes and modifications including, without limitation, those relating to the chemical structures, substituents, derivatives, formulations or methods of the disclosure may be made without departing from the spirit of the disclosure and the scope of the appended claims. Definitions of the variables in the structures in the schemes herein are commensurate with those of corresponding positions in the formulae presented herein.

### Example 1: 50 L Solid-Phase Synthesis of Eteplirsen Crude Drug Substance

### 1. Materials

TABLE 1
Starting Materials

| Material | | | Chemical | Molecular |
|---|---|---|---|---|
| Name | Chemical Name | CAS Number | Formula | Weight |
| Activated A | Phosphoramidochloridic acid, | 1155373-30-0 | C₃₈H₃₇ClN₇O₄P | 722.2 |
| Subunit | N,N-dimethyl-,[6-[6- | | | |
| | (benzoylamino)-9H-purin-9-yl]- | | | |
| | 4-(triphenylmethyl)-2- | | | |
| | morpholinyl]methyl ester | | | |
| Activated | Phosphoramidochloridic acid, | 1155373-31-1 | C₃₇H₃₇ClN₅O₅P | 698.2 |
| C Subunit | N,N-dimethyl-,[6-[4- | | | |
| | (benzoylamino)-2-oxo-1(2H)- | | | |
| | pyrimidinyl]-4- | | | |
| | (triphenylmethyl)-2- | | | |
| | morpholinyl]methyl ester | | | |
| Activated | Propanoic Acid, 2,2-dimethyl-, | 1155309-89-9 | C₅₁H₅₃ClN₇O₇P | 942.2 |
| DPG | 4-[[[9-[6- | | | |
| Subunit | [[[chloro(dimethylamino)phosphinyl]oxy]methyl]- | | | |
| | 4- | | | |
| | (triphenylmethyl)-2- | | | |
| | morpholinyl]-2-[(2- | | | |
| | phenylacetyl)amino]-9H-purin- | | | |
| | 6-yl]oxy]methyl]phenyl ester | | | |
| Activated | Phosphoramidochloridic acid, | 1155373-34-4 | C₃₁H₃₄ClN₄O₅P | 609.1 |
| T Subunit | N,N-dimethyl-,[6-(3,4-dihydro- | | | |
| | 5-methyl-2,4-dioxo-1(2H)- | | | |
| | pyrimidinyl)]-4- | | | |
| | (triphenylmethyl)-2- | | | |
| | morpholinyl]methyl ester | | | |
| Activated | Butanedioic acid, 1- | 1380600-06-5 | C₄₃H₄₇N₃O₁₀ | 765.9 |
| EG3 Tail | [3aR,4S,7R,7aS)-1,3,3a,4,7,7a- | | | |
| | hexahydro-1,3-dioxo-4,7- | | | |
| | methano-2H-isoindol-2-yl] 4- | | | |
| | [2-[2-[2-[[[4-(triphenylmethyl)- | | | |
| | 1- | | | |
| | piperazinyl]carbonyl]oxy]ethoxy]ethoxy]ethyl] | | | |
| | ester | | | |

The term “EG3” refers to triethylene glycol moieties conjugated to the oligomer, e.g., at its 3′- or 5′-end:

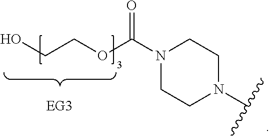

### Chemical Structures of Starting Materials:

### A. Activated EG3 Tail

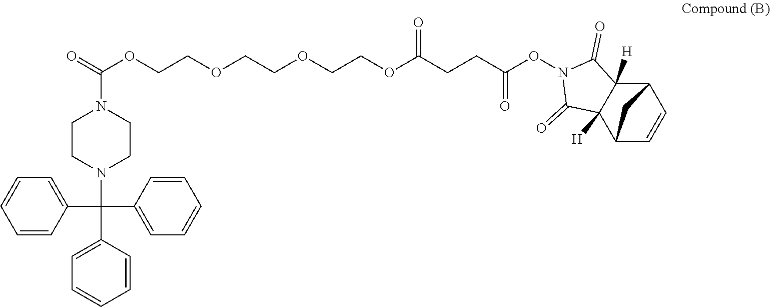 [SMILES: O=C(CCC(=O)ON1C(=O)[C@@H]2[C@H](C1=O)[C@@H]1C=C[C@H]2C1)OCCOCCOCCOC(=O)N1CCN(C(c2ccccc2)(c2ccccc2)c2ccccc2)CC1]

B. Activated C Subunit (for Preparation, See U.S. Pat. No. 8,067,571)

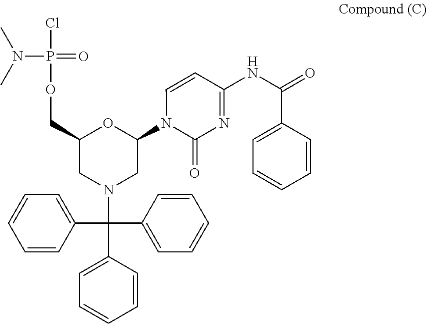 [SMILES: CN(C)P(=O)(Cl)OC[C@@H]1CN(C(c2ccccc2)(c2ccccc2)c2ccccc2)C[C@H](n2ccc(NC(=O)c3ccccc3)nc2=O)O1]

C. Activated a Subunit (for Preparation, See U.S. Pat. No. 8,067,571)

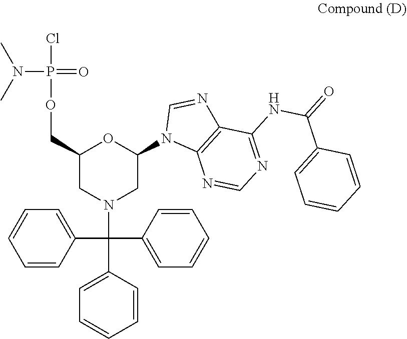 [SMILES: CN(C)P(=O)(Cl)OC[C@@H]1CN(C(c2ccccc2)(c2ccccc2)c2ccccc2)C[C@H](n2cnc3c(NC(=O)c4ccccc4)ncnc32)O1]

### D. Activated DPG Subunit (for Preparation, See WO2009/064471)

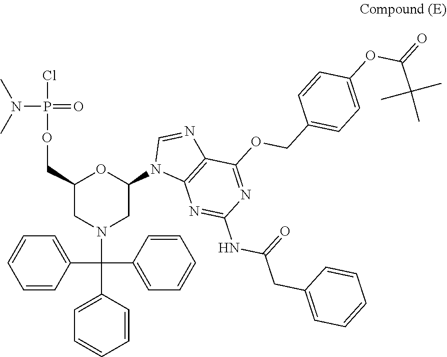 [SMILES: CN(C)P(=O)(Cl)OC[C@@H]1CN(C(c2ccccc2)(c2ccccc2)c2ccccc2)C[C@H](n2cnc3c(OCc4ccc(OC(=O)C(C)(C)C)cc4)nc(NC(=O)Cc4ccccc4)nc32)O1]

### E. Activated T Subunit (for Preparation, See WO 2013/082551)

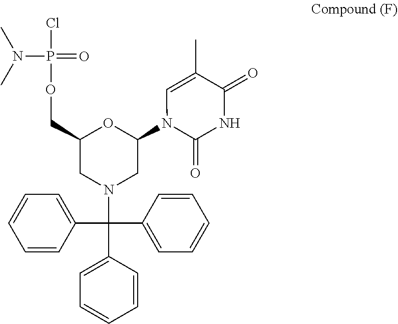 [SMILES: Cc1cn([C@H]2CN(C(c3ccccc3)(c3ccccc3)c3ccccc3)C[C@@H](COP(=O)(Cl)N(C)C)O2)c(=O)[nH]c1=O]

### F. Anchor Loaded Resin

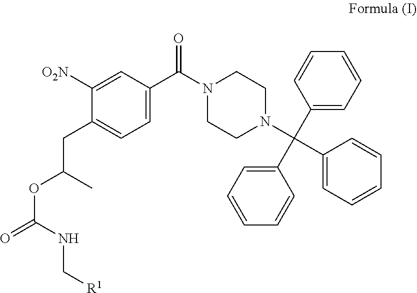

wherein R¹ is a support-medium.

TABLE 2
Description of Solutions for Solid Phase Oligomer Synthesis of
Eteplirsen Crude Drug Substance

| Solution Name | Solution Composition |
|---|---|
| NCP2 Anchor | 37.5 L NMP and 1292 g NCP2 Anchor |
| Solution | |
| DEDC Capping | 4.16 L Diethyl Dicarbonate (DEDC), 3.64 L NEM, |
| Solution | and 33.8 L DCM |
| CYTFA Solution | 2.02 kg 4-cyanopyridine, 158 L DCM, 1.42 L TFA, |
| | 39 L TFE, and 2 L purified water |
| Neutralization | 35.3 L IPA, 7.5 L DIPEA, and 106.5 L DCM |
| Solution | |
| Cleavage Solution | 1,530.04 g DTT, 6.96 L NMP, and 2.98 L DBU |

### 2. Synthesis of Eteplirsen Crude Drug Substance

A. Resin Swelling

750 g of Anchor Loaded Resin and 10.5 L of NMP were charged to a 50 L silanized reactor and stirred for 3 hours. The NMP was drained and the resin was washed twice with 5.5 L each of DCM and twice with 5.5 L each of 30% TFE/DCM.

B. Cycle 0: EG3 Tail Coupling

The resin was washed three times with 5.5 L each of 30% TFE/DCM and drained. 5.5 L of CYTFA Solution for 15 minutes, drained, and repeated with 5.5 L of CYTFA Solution for 15 minutes without draining to which 122 mL of 1:1 NEM/DCM was charged and the suspension stirred for 2 minutes and drained. The resin was washed twice with 5.5 L of Neutralization solution for 5 minutes and drained, then twice with 5.5 L each of DCM and drained. A solution of 706.2 g of activated EG3 Tail (MW 765.85) and 234 mL of NEM in 3 L of DMI was charged to the resin and stirred for 3 hours at RT and drained. The resin was washed twice with 5.5 L each of Neutralization Solution for 5 minutes per each wash, and once with 5.5 L of DCM and drained. A solution of 374.8 g of benzoic anhydride 195 mL NEM in 2680 mL NMP was charged and stirred for 15 minutes and drained. The resin was stirred with 5.5 L of Neutralization Solution for 5 minutes, then washed once with 5.5 L of DCM and twice with 5.5 L each of 30% TFE/DCM. The resin was suspended in 5.5 L of 30% TFE/DCM and held for 14 hours.

C. Subunit Coupling Cycles 1-30

i. Pre-Coupling Treatments

Prior to each coupling cycle as described in Table 3, the resin was: 1) washed with 30% TFE/DCM; 2) a) treated with CYTFA Solution 15 minutes and drained, and b) treated with CYTFA Solution for 15 minutes to which was added 1:1 NEM/DCM, stirred, and drained; 3) stirred three times with Neutralization Solution; and 4) washed twice with DCM. See Table 3.

ii. Post Coupling Treatments

After each subunit solution was drained as described in Table 3, the resin was: 1) washed with DCM; and 2) washed two times with 30% TFE/DCM. If the resin was held for a time period prior to the next coupling cycle, the second TFE/DCM wash was not drained and the resin was retained in said TFE/DCM wash solution. See Table 3.

iii. Activated Subunit Coupling Cycles

The coupling cycles were performed as described in Table 3.

iv. Final IPA Washing

The resin was washed 8 times with 19.5 L each of IPA, dried under vacuum at room temperature for about 63.5 hours to a dried weight of 5,579.8 g.

D. Cleavage

The above resin bound Eteplirsen Crude Drug Substance was divided into two lots, each lot was treated as follows. A 2,789.9 g lot of resin was: 1) stirred with 10 L of NMP for 2 hrs, then the NMP was drained; 2) washed tree times with 10 L each of 30% TFE/DCM; 3) treated with 10 L CYTFA Solution for 15 minutes; and 4) 10 L of CYTFA Solution for 15 minutes to which 130 mL 1:1 NEM/DCM was then added and stirred for 2 minutes and drained. The resin was treated three times with 10 L each of Neutralization Solution, washed six times with 10 L of DCM, and eight times with 10 L each of NMP. The resin was treated with a Cleaving Solution of 1530.4 g DTT and 2980 DBU in 6.96 L NMP for 2 hours to detach the Eteplirsen Crude Drug Substance from the resin. The Cleaving Solution was drained and retained in a separate vessel. The reactor and resin were washed with 4.97 L of NMP which was combined with the Cleaving Solution.

TABLE 3

| | | | | | | | Post-Coupling | |
|---|---|---|---|---|---|---|---|---|
| | Pre-coupling Treatment | | | | | | Treatment | |
| | 1 | | | | Coupling Cycle | | | 2 |
| Cycle | 30% | | 3 | | Quantity | RT | | 30% |
| No.: | TFE/ | 2 | Neutral- | 4 | SU (g) | Coupling | 1 | TFE/ |
| Subunit | DCM | CYTFA | ization | DCM | NEM (L) | Time | DCM | DCM |
| (SU) | Wash | Solution¹ | Solution | Wash | DMI (L) | (Hrs.) | Wash | Wash |
| 1: C | 5.5 L | a) 5.5 L | 3 × 5.5 L | 5.5 L | 536.7 g; | 5 | 5.5 L | 2 × 5.5 L |
| | | b) 5.5 L, | | | 195 ml NEM; | | | |
| | | 122 ml | | | 3.2 L DMI | | | |
| 2: T | 7.0 L | a) 7 L | 3 × 7 L | 2 × 7 L | 468.2 g and | 4.25 | 7 L | 2 × 7 L² |
| | | b) 7 L, | | | 195 ml NEM | | | |
| | | 158 ml | | | 3.2 L DMI | | | |
| 3: C | 8 L | a) 8 L | 3 × 8 L | 2 × 8 L | 536.7 g; | 4.25 | 8 L | 2 × 8 L |
| | | b) 8 L, | | | 195 ml NEM; | | | |
| | | 182 ml | | | 3.4 L DMI | | | |
| 4: C | 9 L | a) 9 L | 3 × 9 L | 2 × 9 L | 536.7 g; | 4.25 | 9 L | 2 × 9 L³ |
| | | b) 9 L, | | | 195 ml NEM; | | | |
| | | 206 ml | | | 3.6 L DMI | | | |
| 5: A | 9.5 L | a) 9.5 L | 3 × 9.5 L | 2 × 9.5 L | 555.2 g; | 4.25 | 9.5 L | 2 × 9.5 L |
| | | b) 9.5 L, | | | 195 ml NEM; | | | |
| | | 220 ml | | | 3.4 L DMI | | | |
| 6: A | 10 L | a) 10 L | 3 × 10 L | 2 × 10 L | 555.2 g; | 4.25 | 10 L | 2 × 10 L⁴ |
| | | b) 10 L, | | | 195 ml NEM; | | | |
| | | 232 ml | | | 3.45 L DMI | | | |
| | | | | | | | Post-Coupling | |
| | Pre-coupling Treatment | | | | | | Treatment | |
| | 1 | | | | Coupling Cycle | | | 2 |
| Cycle | 30% | | 3 | | Quantity | RT | | 30% |
| No.: | TFE/ | 2 | Neutral- | 4 | SU (g) | Coupling | 1 | TFE/ |
| Subunit | DCM | CYTFA | ization | DCM | NEM (L) | Time | DCM | DCM |
| (SU) | Wash | Solution | Solution | Wash | DMI (L) | (Hrs.) | Wash | Wash |
| 7: C | 11 L | a) 11 L | 3 × 11 L | 2 × 11 L | 536.7 g; | 4.25 | 11 L | 2 × 11 L |
| | | b) 11 L, | | | 195 ml NEM; | | | |
| | | 256 ml | | | 3.57 L DMI | | | |
| 8: A | 11 L | a) 11 L | 3 × 11 L | 2 × 11 L | 555.2 g; | 4.25 | 11 L | 2 × 11 L⁵ |
| | | b) 11 L, | | | 195 ml NEM; | | | |
| | | 256 ml | | | 3.64 L DMI | | | |
| 9: T | 11.5 L | a) 11.5 L | 3 × 11.5 L | 2 × 11.5 L | 468.2 g; | 4.25 | 11.5 L | 2 × 11.5 L |
| | | b) 11.5 L | | | 195 ml NEM; | | | |
| | | 268 ml | | | 3.72 L DMI | | | |
| 10: C | 12 L | a) 12 L | 3 × 12 L | 2 × 12 L | 536.7 g; | 4.25 | 12 L | 2 × 12 L⁶ |
| | | b) 12 L, | | | 195 ml NEM; | | | |
| | | 280 ml | | | 3.96 L DMI | | | |
| 11: A | 13.5 L | a) 13.5 L | 3 × 13.5 L | 2 × 13.5 L | 721.7 g; | 4.25 | 13.5 L | 2 × 13.5 L |
| | | b) 13.5 L, | | | 253 ml NEM; | | | |
| | | 204 ml | | | 4.02 L DMI | | | |
| 12: A | 13.5 L | a) 13.5 L | 3 × 13.5 L | 2 × 13.5 L | 721.7 g; | 4.25 | 13.5 L | 2 × 13.5 L⁷ |
| | | b) 13.5 L, | | | 253 ml NEM; | | | |
| | | 204 ml | | | 4.02 L DMI | | | |
| 13: DPG | 14 L | a) 14 L | 3 × 14 L | 2 × 14 L | 941.9 g; | 4.25 | 14 L | 2 × 14 L |
| | | b) 14 L, | | | 253 ml NEM; | | | |
| | | 216 ml | | | 4.02 L DMI | | | |
| 14: DPG | 14.5 L | a) 14.5 L | 3 × 14.5 L | 2 × 14.5 L | 941.9 g; | 4.25 | 14.5 L | 2 × 14.5 L⁸ |
| | | b) 14.5 L, | | | 253 ml NEM; | | | |
| | | 228 ml | | | 4.1 L DMI | | | |
| 15: A | 15.5 L | a) 15.5 L | 3 × 15.5 L | 2 × 15.5 L | 721.7 g; | 4.25 | 15.5 L | 2 × 15.5 L |
| | | b) 15.5 L, | | | 253 ml NEM; | | | |
| | | 254 ml | | | 4.26 L DMI | | | |
| 16: A | 15.5 L | a) 15.5 L | 3 × 15.5 L | 2 × 15.5 L | 721.7 g; | 4.25 | 15.5 L | 2 × 15.5 L⁹ |
| | | b) 15.5 L, | | | 253 ml NEM; | | | |
| | | 254 ml | | | 4.26 L DMI | | | |
| 17: DPG | 16 L | a) 16 L | 3 × 16 L | 2 × 16 L | 941.9 g; | 4.75 | 16 L | 2 × 16 L |
| | | b) 16 L, | | | 253 ml NEM; | | | |
| | | 366 ml | | | 4.4 L DMI | | | |
| 18: A | 16.5 L | a) 16.5 | 3 × 16.5 L | 2 × 16.5 L | 721.7 g; | 4.25 | 16.5 L | 2 × 16.5 L¹⁰ |
| | | b) 16.5 L, | | | 253 ml NEM; | | | |
| | | 378 ml | | | 4.4 L DMI | | | |
| 19: T | 16.5 L | a) 16.5 L | 3 × 16.5 L | 2 × 16.5 L | 608.7 g; | 4.25 | 16.5 L | 2 × 16.5 L |
| | | b) 16.5 L, | | | 253 ml NEM; | | | |
| | | 378 ml | | | 4.57 L DMI | | | |
| 20: DPG | 17 L | a) 17 L | 3 × 17 L | 2 × 17 L | 941.9 g; | 4.75 | 17 L | 2 × 17 L¹¹ |
| | | b) 17 L, | | | 253 ml NEM; | | | |
| | | 390 ml | | | 4.57 L DMI | | | |
| 21: DPG | 17 L | a) 17 L | 3 × 17 L | 2 × 17 L | 1159.2 g; | 4.25 | 17 L | 2 × 17 L |
| | | b) 17 L, | | | 311 ml NEM; | | | |
| | | 390 ml | | | 4.72 L DMI | | | |
| 22: C | 17.5 L | a) 17.5 L | 3 × 17.5 L | 2 × 17.5 L | 858.7 g; | 4.75 | 17.5 L | 2 × 17.5 L¹² |
| | | b) 17.5 L, | | | 311 ml NEM; | | | |
| | | 402 ml | | | 4.72 L DMI | | | |
| 23: A | 17.5 L | a) 17.5 L | 3 × 17.5 L | 2 × 17.5 L | 888.3 g; | 4.25 | 17.5 L | 2 × 17.5 L |
| | | b) 17.5 L, | | | 311 ml NEM; | | | |
| | | 402 ml | | | 4.88 L DMI | | | |
| 24: T | 18 L | a) 18 L | 3 × 18 L | 2 × 18 L | 749.1 g; | 4.25 | 18 L | 2 × 18 L¹³ |
| | | b) 18 L, | | | 311 ml NEM; | | | |
| | | 414 ml | | | 4.95 L DMI | | | |
| 25: T | 18 L | a) 18 L | 3 × 18 L | 2 × 18 L | 749.1 g; | 4.25 | 18 L | 2 × 18 L |
| | | b) 18 L, | | | 311 ml NEM; | | | |
| | | 414 ml | | | 5.1 L DMI | | | |
| 26: T | 18.5 L | a) 18.5 L | 3 × 18.5 L | 2 × 18.5 L | 749.1 g; | 4.25 | 18.5 L | 2 × 18.5 L¹⁴ |
| | | b) 18.5 L, | | | 311 ml NEM; | | | |
| | | 426 ml | | | 5.1 L DMI | | | |
| 27: C | 18.5 L | a) 18.5 L | 3 × 18.5 L | 2 × 18.5 L | 858.7 g; | 4.25 | 18.5 L | 2 × 18.5 L |
| | | b) 18.5 L, | | | 311 ml NEM; | | | |
| | | 426 ml | | | 5.25 L DMI | | | |
| 28: T | 19 L | a) 19 L | 3 × 19 L | 2 × 19 L | 749.1 g; | 4.25 | 19 L | 2 × 19 L¹⁵ |
| | | b) 19 L, | | | 311 ml NEM; | | | |
| | | 438 ml | | | 5.25 L DMI | | | |
| 29: A | 19 L | a) 19 L | 3 × 19 L | 2 × 19 L | 888.3 g; | 4.25 | 19 L | 2 × 19 L |
| | | b) 19 L, | | | 311 ml NEM; | | | |
| | | 438 ml | | | 5.41 L DMI | | | |
| 30: DPG | 19.5 L | a) 19.5 L | 3 × 19.5 L | 2 × 19.5 L | 1159.2 g; | 4.75 | 19.5 L | 2 × 19.5 L |
| | | b) 19.5 L, | | | 311 ml NEM; | | | |
| | | 450 ml | | | 5.44 L DMI | | | |

¹ml indicates the amount of 1:1 NEM/DCM
²Resin held at this step for ½ day
³Resin held at this step for ½ day
⁴Resin held at this stage for 0.4 days
⁵Resin held at this stage for 2.5 days
⁶Resin held at this stage for ½ day
⁷Resin held at this stage for 0.4 days
⁸Resin held at this stage for 0.4 days
⁹Resin held at this stage for 0.4 days
¹⁰Resin held at this stage for 1.5 days
¹¹Resin held at this stage for 0.3 days
¹²Resin held at this stage for 0.4 days
¹³Resin held at this stage for 0.4 days
¹⁴Resin held at this stage for 0.4 days
¹⁵Resin held at this stage for 0.3 days

E. Deprotection

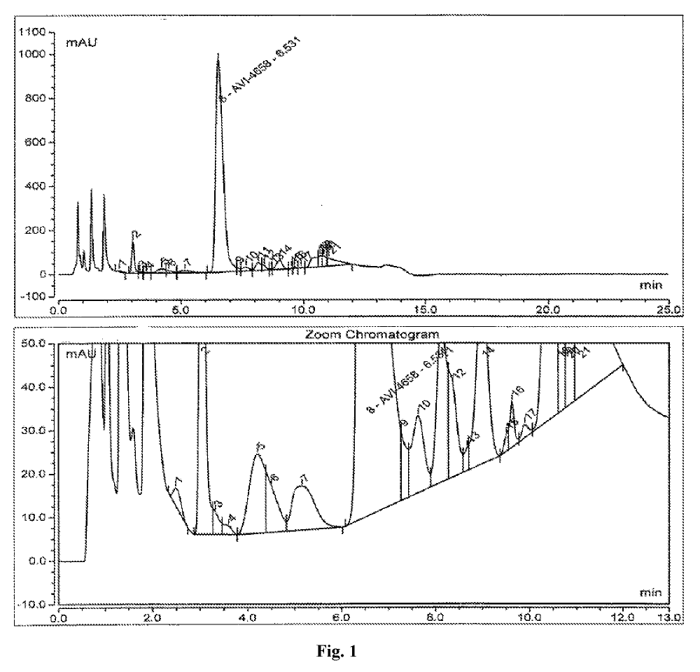

The combined Cleaving Solution and NMP wash were transferred to a pressure vessel to which was added 39.8 L of NH₄OH that had been chilled in a −10° to −25° C. in a freezer. The pressure vessel was sealed and heated to 45° C. for 16 hrs then allowed to cool to 25° C. The deprotection solution containing the Eteplirsen crude drug substance was diluted 3:1 with purified water and pH adjusted to 3.0 with 2M phosphoric acid, then to pH 8.03 with NH₄OH. HPLC (C18) 73-74% (FIG. 1).

TABLE 4
Data of FIG. 1

| | | Reten- | | | | |
|---|---|---|---|---|---|---|
| | Com- | tion | Rel. | | Rel. | |
| | pound | Time | Ret. Time. | Area | Area | Plates |
| Peak # | Name | (min) | Product | {mAu*min) | % | (USP) |
| 1 | | 2.488 | 0.381 | 0.821928 | 0.18 | 1105 |
| 2 | | 3.047 | 0.467 | 17.661449 | 3.91 | 4047 |
| 3 | | 3.324 | 0.509 | 0.818258 | 0.18 | n.a. |
| 4 | | 3.605 | 0.552 | 0.465598 | 0.10 | 7 |
| 5 | | 4.213 | 0.645 | 6.558899 | 1.45 | 301 |
| 6 | | 4.504 | 0.690 | 3.324238 | 0.74 | 191690 |
| 7 | | 5.160 | 0.790 | 5.644073 | 1.25 | 651 |
| 8 | AVI- | 6.531 | 1.000 | 332.238891 | 73.47 | 2313 |
| | 4658 | | | | | |
| 9 | | 7.269 | 1.113 | 2.063159 | 0.46 | n.a. |
| 10 | | 7.643 | 1.170 | 5.556411 | 1.23 | 2734 |
| 11 | | 8.139 | 1.246 | 8.711530 | 1.93 | 3572 |
| 12 | | 8.382 | 1.283 | 4.654783 | 1.03 | 1835 |
| 13 | | 8.678 | 1.329 | 0.562426 | 0.12 | n.a. |
| 14 | | 9.009 | 1.379 | 12.031923 | 2.66 | 6078 |
| 15 | | 9.500 | 1.455 | 0.385563 | 0.09 | n.a. |
| 16 | | 9.626 | 1.474 | 1.171507 | 0.26 | 46084 |
| 17 | | 9.898 | 1.516 | 0.484362 | 0.11 | 21328 |
| 18 | | 10.598 | 1.623 | 14.589918 | 3.23 | n.a. |
| 19 | | 10.680 | 1.635 | 7.520577 | 1.66 | 918 |
| 20 | | 10.811 | 1.656 | 8.604558 | 1.90 | 296 |
| 21 | | 11.045 | 1.691 | 18.351689 | 4.06 | 49919 |

### Example 2: Purification of Eteplirsen Crude Drug Substance

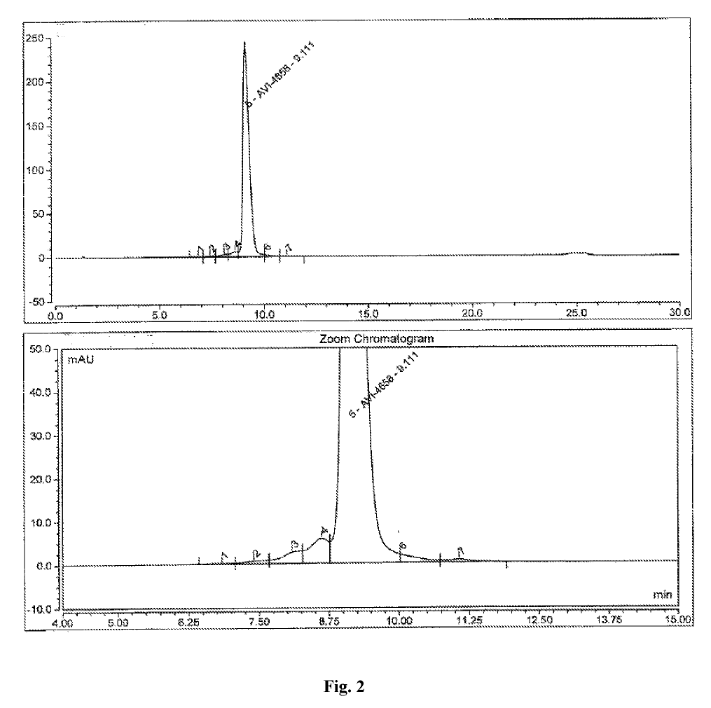

The deprotection solution from Example 1 containing the Eteplirsen crude drug substance was loaded onto a column of ToyoPearl Super-Q 650S anion exchange resin (Tosoh Bioscience) and eluted with a gradient of 0-35% B over 17 column volume (Buffer A: 10 mM sodium hydroxide; Buffer B: 1 M sodium chloride in 10 mM sodium hydroxide) and fractions of acceptable purity (C18 and SCX HPLC) were pooled to a purified drug product solution. HPLC: 97.74% (C18) 94.58% (SCX; FIG. 2).

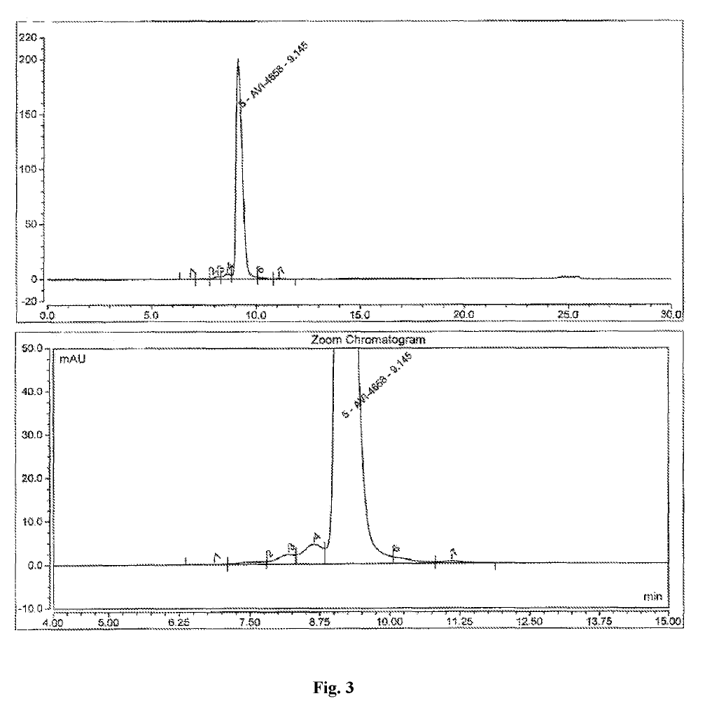

The purified drug substance solution was desalted and lyophilized to 1959 g purified Eteplirsen drug substance. Yield 61.4%; HPLC: 97.7% (C18) 94.6% (SCX; FIG. 3).

TABLE 5
Data of FIG. 2

| | | Reten- | | | | |
|---|---|---|---|---|---|---|
| | Com- | tion | Rel. | | | |
| | pound | Time | Ret. Time. | Area | Area | Plates |
| Peak # | Name | (min) | (Product) | {mAu*min) | Percent | (USP) |
| 1 | | 6.837 | 0.750 | 0.050757 | 0.058 | 41574 |
| 2 | | 7.405 | 0.813 | 0.303271 | 0.344 | 841 |
| 3 | | 8.086 | 0.887 | 1.130007 | 1.280 | 13 |
| 4 | | 8.615 | 0.946 | 2.265128 | 2.567 | 761 |
| 5 | AVI- | 9.111 | 1.000 | 83.468700 | 94.583 | 4405 |
| | 4658 | | | | | |
| 6 | | 10.019 | 1.100 | 0.704599 | 0.798 | n.a. |
| 7 | | 11.069 | 1.215 | 0.326550 | 0.370 | 3044 |

TABLE 6
Data of FIG. 3

| | | Reten- | | | | |
|---|---|---|---|---|---|---|
| | Com- | tion | Rel. | | | |
| | pound | Time | Ret. Time. | Area | Area | Plates |
| Peak # | Name | (min) | (Product) | {mAu*min) | Percent | (USP) |
| 1 | | 6.866 | 0.751 | 0.044399 | 0.063 | 608 |
| 2 | | 7.794 | 0.852 | 0.280589 | 0.397 | n.a. |
| 3 | | 8.188 | 0.895 | 0.816793 | 1.156 | 209 |
| 4 | | 8.644 | 0.945 | 1.842896 | 2.608 | 1147 |
| 5 | AVI | 9.145 | 1.000 | 66.857088 | 94.622 | 4664 |
| | 4658 | | | | | |
| 6 | | 10.058 | 1.100 | 0.575793 | 0.815 | n.a. |
| 7 | | 11.103 | 1.214 | 0.239454 | 0.339 | 4375 |

TABLE 7
Acronyms

| Acronym | Name |
|---|---|
| DBU | 1,8-Diazabicycloundec-7-ene |
| DCM | Dichloromethane |
| DIPEA | N,N-Diisopropylethylamine |
| DMI | 1,3-Dimethyl-2-imidazolidinone |
| DTT | Dithiothreitol |
| IPA | Isopropyl alcohol |
| MW | Molecular weight |
| NEM | N-Ethylmorpholine |
| NMP | N-Methyl-2-pyrrolidone |
| RT | Room temperature |
| TFA | 2,2,2-Trifluoroacetic acid |
| TFE | 2,2,2-Trifluoroethanol |

### Example 3: Exemplary Eteplirsen Pharmaceutical Compositions

TABLE 8
Exemplary Composition 1: Total volume of 1 mL

| | |
|---|---|
| Eteplirsen | 50 mg |
| sodium chloride, USP | 8 mg |
| potassium chloride, USP | 0.2 mg |
| potassium phosphate monobasic, NF | 0.2 mg |
| sodium phosphate dibasic anhydrous, USP | 1.14 mg |
| Water for injection, USP | q.s. |

Exemplary Composition 2: Total volume of 2 mL

| | |
|---|---|
| Eteplirsen | 100 mg |
| sodium chloride, USP | 16 mg |
| potassium chloride, USP | 0.4 mg |
| potassium phosphate monobasic, NF | 0.4 mg |
| sodium phosphate dibasic anhydrous, USP | 2.28 mg |
| water for injection, USP | q.s. |

Exemplary Composition 3: Total volume of 10 mL

| | |
|---|---|
| Eteplirsen | 500 mg |
| sodium chloride, USP | 80 mg |
| potassium chloride, USP | 2 mg |
| potassium phosphate monobasic, NF | 2 mg |
| sodium phosphate dibasic anhydrous, USP | 11.4 mg |
| water for injection, USP | q.s. |

Exemplary Composition 4: Total volume of 1 mL

| | |
|---|---|
| Eteplirsen | 50 mg |
| sodium chloride | 8 mg |
| potassium chloride | 0.2 mg |
| potassium phosphate monobasic | 0.2 mg |
| sodium phosphate dibasic anhydrous | 1.14 mg |
| Water for injection | q.s. |

Exemplary Composition 5: Total volume of 2 mL

| | |
|---|---|
| Eteplirsen | 100 mg |
| sodium chloride | 16 mg |
| potassium chloride | 0.4 mg |
| potassium phosphate monobasic | 0.4 mg |
| sodium phosphate dibasic anhydrous | 2.28 mg |
| water for injection | q.s. |

Exemplary Composition 6: Total volume of 10 mL

| | |
|---|---|
| Eteplirsen | 500 mg |
| sodium chloride | 80 mg |
| potassium chloride | 2 mg |
| potassium phosphate monobasic | 2 mg |
| sodium phosphate dibasic anhydrous | 11.4 mg |
| water for injection | q.s. |

For each of Exemplary Compositions 1-6 (Table 8), the amount of water present is sufficient to achieve a concentration of Eteplirsen of about 50 mg/mL of the pharmaceutical composition. Further, the pH of the Exemplary Compositions is about 7.5, and the osmolality of the Exemplary Compositions ranges from about 260 mOsm to about 320 mOsm.

### INCORPORATION BY REFERENCE

The contents of all references (including literature references, issued patents, published patent applications, and co-pending patent applications) cited throughout this application are hereby expressly incorporated herein in their entireties. Unless otherwise defined, all technical and scientific terms used herein are accorded the meaning commonly known to one with ordinary skill in the art.

### EQUIVALENTS

Those skilled in the art will recognize, or be able to ascertain using no more than routine experimentation, many equivalents of the specific embodiments of the disclosure described herein. Such equivalents are intended to be encompassed by the following claims.

1. A pharmaceutical composition, comprising:
a) Eteplirsen;
b) sodium chloride;
c) potassium chloride;
d) potassium phosphate monobasic;
e) sodium phosphate dibasic; and
f) water,
wherein the concentration of Eteplirsen is about 50 mg/mL of the pharmaceutical composition.

2. The pharmaceutical composition of claim 1, comprising:
a) 40-60 mg of Eteplirsen;
b) 6.4-9.6 mg of sodium chloride;
c) 0.16-0.24 mg of potassium chloride;
d) 0.16-0.24 mg of potassium phosphate monobasic;
e) 0.91-1.37 mg of sodium phosphate dibasic; and
f) water,
wherein the concentration of Eteplirsen is about 50 mg/mL of the pharmaceutical composition.

3. The pharmaceutical composition of claim 2, comprising about 50 mg of Eteplirsen.

4. The pharmaceutical composition of claim 1, comprising:
a) about 50 mg of Eteplirsen;
b) about 8 mg of sodium chloride;
c) about 0.2 mg of potassium chloride;
d) about 0.2 mg of potassium phosphate monobasic;
e) about 1.14 mg of sodium phosphate dibasic; and
f) water,
wherein the concentration of Eteplirsen is about 50 mg/mL of the pharmaceutical composition.

5. The pharmaceutical composition of claim 1, comprising:
a) 80-120 mg of Eteplirsen;
b) 12.8-19.2 mg of sodium chloride;
c) 0.32-0.48 mg of potassium chloride;
d) 0.32-0.48 mg of potassium phosphate monobasic;
e) 1.02-1.54 mg of sodium phosphate dibasic; and
f) water,
wherein the concentration of Eteplirsen is about 50 mg/mL of the pharmaceutical composition.

6. The pharmaceutical composition of claim 5, comprising about 100 mg of Eteplirsen.

7. The pharmaceutical composition of claim 5, comprising:
a) about 100 mg of Eteplirsen;
b) about 16 mg of sodium chloride;
c) about 0.4 mg of potassium chloride;
d) about 0.4 mg of potassium phosphate monobasic;
e) about 2.28 mg of sodium phosphate dibasic; and
f) water,
wherein the concentration of Eteplirsen is about 50 mg/mL of the pharmaceutical composition.

8. The pharmaceutical composition of claim 1, comprising:
a) 400-600 mg of Eteplirsen;
b) 64-96 mg of sodium chloride;
c) 1.6-2.4 mg of potassium chloride;
d) 1.6-2.4 mg of potassium phosphate monobasic;
e) 9.0-14.0 mg of sodium phosphate dibasic; and
f) water,
wherein the concentration of Eteplirsen is about 50 mg/mL of the pharmaceutical composition.

9. The pharmaceutical composition of claim 8, comprising about 500 mg of Eteplirsen.

10. The pharmaceutical composition of claim 8, comprising:
a) about 500 mg of Eteplirsen;
b) about 80 mg of sodium chloride;
c) about 2 mg of potassium chloride;
d) about 2 mg of potassium phosphate monobasic;
e) about 11.4 mg of sodium phosphate dibasic; and
f) water,
wherein the concentration of Eteplirsen is about 50 mg/mL of the pharmaceutical composition.

11. The pharmaceutical composition of claim 1, comprising:
a) about 5 w/v % Eteplirsen;
b) about 0.8 w/v % sodium chloride;
c) about 0.02 w/v % potassium chloride;
d) about 0.02 w/v % potassium phosphate monobasic;
e) about 0.114 w/v % sodium phosphate dibasic; and
f) water.

12. The pharmaceutical composition of claim 1, comprising:
a) about 50 mg/mL Eteplirsen;
b) about 8 mg/mL sodium chloride;
c) about 0.2 mg/mL potassium chloride;
d) about 0.2 mg/mL potassium phosphate monobasic;
e) about 1.14 mg/mL sodium phosphate dibasic; and
f) water.

13. The pharmaceutical composition according to any one of claims 1-12, wherein the pH of the pharmaceutical composition is about 7.5, and the osmolality of the pharmaceutical composition ranges from about 260 mOsm to about 320 mOsm.

14. A method for treating Duchenne muscular dystrophy (DMD) in a subject in need thereof wherein the subject has a mutation of the dystrophin gene that is amenable to exon 51 skipping, comprising administering to the subject a pharmaceutical composition of any one of claims 1-13.

15. Use of the pharmaceutical composition of any one of claims 1-13 for the manufacture of a medicament for the treatment Duchenne muscular dystrophy (DMD) in a subject in need thereof, wherein the subject has a mutation of the dystrophin gene that is amenable to exon 51 skipping.


SEQUENCE LISTING (2 sequences)

SEQ ID NO: 1 - DNA, 30 bp, Artificial Sequence
  ETEPLIRSEN PMO AVI-4658
ctccaacatcaaggaagatggcatttctag

SEQ ID NO: 2 - RNA, 30 nt, Artificial Sequence
  Synthetic
cuccaacaucaaggaagauggcauuucuag